(19) United States
(12) Patent Application Publication
(10) Pub. No.: US 2025/0110115 A1
(43) Pub. Date: Apr. 3, 2025

(54) MEANS AND METHODS FOR DETERMINING CELLULAR AVIDITY

(71) Applicant: LUMICKS CA HOLDING B.V., Amsterdam (NL)

(72) Inventors: Marinus Theodorus Johannes VAN LOENHOUT, Amsterdam (NL); Serena Alba DAVOLI, Amsterdam (NL); Kieren Jay PATEL, Amsterdam (NL)

(21) Appl. No.: 18/865,708

(22) PCT Filed: May 31, 2023
(86) PCT No.: PCT/EP2023/064566

§ 371 Date: Nov. 14, 2024

(30) Foreign Application Priority Data
Jun. 1, 2022 (EP) 22176826.0
Jun. 1, 2022 (EP) 22180225.9
Dec. 29, 2022 (EP) 22217118.3

(51) Int. Cl. G01N 33/50

(52) U.S. Cl. CPC G01N 33/5032; G01N 33/505

(57) ABSTRACT

The current invention relates to cell-cell interaction and in particular to cellular avidity. Provided are improved means and methods to study cell-cell interaction and characterizing cellular avidity. More in particular, the methods involve studying cell-cell interactions and applying forces in order to break cell-cell bonds in a controllable fashion. In particular, the current inventors provide for means and methods with which highly reliable and consistent cellular avidity experiments can be performed in a high-throughput fashion.

### INTRODUCTION

Despite tremendous clinical success of T cell therapy, including CAR-T cell therapy, for haematological malignancies, many obstacles still remain for the therapy in treatment of a wider range of cancers. Predicting success of T cell therapy using in vitro data alone is still a challenge. Affinity of a binding molecule used for CAR generation towards the target antigen is frequently considered during the in vitro testing, but this does not define CAR-T cell activity. With the increasing complexity of constructing novel methodologies to further improve the clinical outcome of immunotherapies, screening methods to quickly identify the best lead candidates are becoming even more essential. To date, it has become evident that merely measuring the affinity between a CAR and its target will not accurately predict in vitro and in vivo outcomes. On the contrary, cellular avidity, the overall cellular binding strength, can provide a more complete and physiologically relevant measurement that reflects the bona fide interaction between T cells and target cells.

Cellular avidity measurements may better predict cellular responses in vitro, and drive better, more informed decisions at earlier stages for drug selection and potentially improve clinical outcomes. One of the main obstacles in the process of measuring avidity as being a critical parameter is the lack of fast, specific, and accurate tools to assess cellular avidity. The z-Movi® Cell Avidity Analyzer, a platform for measuring cell-cell binding strength, facilitates a direct analysis of CAR-T cells either against surface immobilised antigens or a monolayer of target cells. Using this system, cell-cell interactions are perturbed using resonant sound waves generated by a piezoelectric element and a cell tracking system is employed to measure the required disruption force. This way, cellular avidity measurements can be done which provide for a cellular avidity score which allows to compare e.g. different candidate receptors,

The current inventors, in working with cellular avidity were now looking for means and methods to further improve cellular avidity measurements, in particular in a high throughput format.

### SUMMARY OF THE INVENTION

The current inventors now provide for new and improved methods for assessing cellular avidity. In particular, the current inventors provide for means and methods with which highly reliable and consistent cellular avidity experiments can be performed in a high-throughput fashion which utilize the application of a force away from attached cells. These means and methods include avidity sample plates with one or more wells with which in a highly controllable fashion measurements can be conducted, which includes multiplexing, e.g. combining different samples and/or across multiple wells. By providing a cellular avidity measuring system, which utilizes said avidity sample plate, the methods in accordance with the invention can highly efficiently be performed. This is in stark contrast to means and methods known in the art, which do not allow for high throughput measurements in a highly controllable fashion.

### FIGURES

FIG. 1. Schematic showing a well design of an avidity sample plate. The upper panel shows a single well, with an inlet and an outlet. Depicted in black is the well, e.g. made of a suitable polymer. Depicted at the well surface is antifouling coating, in grey, with a coating for attachment of cells in dark grey, at which target cells are attached (rounded shape in dark grey). The orientation of the well is in upright position. In the well, fluid is contained, in this case a suspension of effector cells depicted as round shapes in lighter grey. The middle panel shows multiple wells positioned on an avidity sample plate in an upright position, the lower panel shows the sample plate in upside down position. In between the upper and lower panel, a pictogram is shown, indicating changing the position from upright to upside down position (or vice versa).

FIG. 2. Schematic depicting method steps in accordance with the invention. The well design and schematic representation thereof as shown in FIG. 1 is used in this flow scheme. In (1) an empty well is shown with a ceiling surface coated for attachment of target cells and anti-fouling coating in the upright position. Target cells are added to the well (2), and the avidity sample plate is placed in the upside down position (3) and the cells are allowed to settle (4) and attach. The avidity sample plate is placed in upright position (5) and target cells that attached to the ceiling surface coated for attachment remain, whereas target cells that did not attach and/or settled at antifouling coated areas in (4) do not remain and settle down in (5), which may include a centrifugation step. Target cells that did not attach may be removed (6) and an image can be captured of the target cells attached to the ceiling surface either in upright position (6) or in upside down position (7), with as depicted to the right the microscope positioned below the well. Next, effector cells are introduced in the well (8), and the effector cells are allowed to settle down to the bottom of the well (9). The avidity sample plate is placed in upside down position (10) and the effector cells now settle down on the target cells (11). An image may be captured of the effector cells that interact with the target cells (11). The avidity sample plate is placed in upright position (12) after a defined time. Effector cells that did not bind with the target cells settle down to the bottom of the well (13), which effector cells may be removed or collected from the well (14). An image may be captured of the effector cells bound to the target cells either in upright position (14) or upside down position (15). The avidity sample plate is placed in upright position (16) and centrifuged, wherein the force exerted can result in effector cells becoming unbound (17), wherein unbound cells may be collected or removed from the well (18). After the centrifugation step, an image may be captured of the effector cells that remained bound to the target cells in the upright position (18) or upside down position (19). Steps (17)-(19) may be repeated with increasing centrifugation forces.

FIG. 3. A cross section of a well design suitable for an avidity sample plate. A) Depicted is a pipet (1) which introduces liquid at an inlet channel (2). Depicted in black are cells that are attached to the ceiling surface. This design includes a reservoir (3) which allows for more stable cell culture conditions, reduction of osmotic shifts with evaporation, or reduce risk of drying out of the channel. The outlet channel (4) has a short diffusion distance from the ceiling surface to the reservoir which will allow for efficient gas exchange which is useful to accommodate appropriate cell culture conditions. The well design allows to accommodate different fluid levels (5) and (7), which may be varied (6) which is useful e.g. for introducing fluid in the well and/or removing fluids therefrom in the methods of the invention. B) on the left, the well in the upright position is shown, on the right, in the upside down position. The surface tension and capillary forces of the fluid at the inlet and outlet channels (depicted in black) allow to retain the fluid in the wells.

FIG. 4. schematically shows a well 300 (comprised at an avidity sample plate) in cross section, having a flow channel 310 extending between a channel inlet 320 and a channel outlet 320 at an opening side and defining a ceiling 312 and a floor 314 associated with the opening side OS. In an upright orientation of the sample holder the opening side OS is upward. Thus, the channel can be loaded via the inlet 320 from above. In A) the channel is loaded with liquid and target cells 330. In B) the avidity sample plate is flipped, and a layer of target cells is formed. In C) the well is flipped back in upright position, washed, and inserted in a centrifuge holder, and in D), subsequently, a centrifugation force is exerted keeping air bubbles out.

### DETAILED DESCRIPTION

Accordingly, the current invention provides for means and methods of assessing cellular avidity of an effector cell and a target cell. In one embodiment, a method is provided of assessing cellular avidity of an effector cell and a target cell comprising the steps of:
a) providing target cells at a defined concentration;
b) optionally, staining the target cells;
c) providing an avidity sample plate with one or more wells with a ceiling surface coated for attachment of target cells, wherein optionally at least the bottom surface is coated with anti-fouling, wherein the avidity sample plate is optionally provided with an identifier, and, optionally provided with one or more sensors;
d) introducing the target cells, homogenized, to one or more wells of the avidity sample plate for obtaining a monolayer of target cells, with the avidity sample plate in the upright position;
e) placing the avidity sample plate in an upside down position, thereby allowing the target cells to settle to the ceiling surface and attach thereto to form a monolayer;
f) optionally, culturing the target cells, with the avidity sample plate in either upside down position or upright position, in a controlled incubator;
g) optionally, blocking the ceiling surface with attached target cells with a blocking agent;
h) placing the avidity sample plate in the upright position, optionally in a centrifuge and centrifuge to spin down target cells that did not attach to the ceiling surface;
i) optionally, washing the cells with the avidity sample plate in the upright position, to remove target cells that did not attach to the ceiling surface;
j) optionally, acquire an image of the monolayer of target cells attached to the ceiling surface.
k) optionally, checking the quality of monolayer of target cells based on the image of the monolayer;
l) providing effector cells at a defined concentration;
m) optionally, and preferably, staining the effector cells;
n) introducing the effector cells, homogenized, to one or more wells of the avidity sample plate in the upright position and optionally allowing the effector cells to settle to the bottom of the avidity sample plate;
o) placing the avidity sample plate in the upside down position to allow the effector cells to settle on the monolayer of target cells attached to the ceiling of the avidity sample plate and incubating the effector cells with the target cells, for a defined time;
p) optionally, capturing, with the avidity sample plate in the upside down position, an image of the effector cells and the monolayer of target cells;
q) placing the avidity sample plate in the upright position;
r) optionally, allowing the effector cells that did not bind to the target cells to settle to the bottom of the avidity sample plate, and optionally, collecting or washing away said effector cells that settled to the bottom;
s) optionally, capturing an image of the effector cells bound to the monolayer of target cells, with the avidity sample plate in the upright position;
t) placing the avidity sample plate in the upright position in the centrifuge and centrifuging, at a defined centrifugal force, e.g. 1000×g, for a defined time;
u) capturing an image of the effector cells bound to the monolayer of target cells, with the avidity sample plate in the upright position;
v) optionally, collecting or washing away effector cells that become unbound from the target cells, before or after capturing the image;
w) optionally, repeating steps t) to v), one or more times, with for each consecutive time an increasing centrifugal force;
x) optionally, collecting the effector cells that remain bound to the target cells after the image was captured.

As described herein, effector cells include effector cells of the immune system that can exert an effect, via a receptor. For example, a T cell carrying a T cell receptor can bind an antigen on a cancer cell, upon which it can e.g. exert a cytotoxic effect and kill the target cell. Effector cells can be derived from nature, e.g. obtained from a host, e.g. a human, and can also include genetically modified cells wherein e.g. a receptor in particular useful is provided to an effector cell.

Target cells in accordance with the invention are the cells on which the effector cells are to exert an effect, e.g. bind therewith and trigger an immune reaction thereto. Target cells include cancer cells presenting an antigen. An antigen may be presented by MHC, i.e. HLA in humans, which are specialized receptors that present peptides e.g. derived from digested proteins expressed by the cell (e.g. usually 8-11 amino acids in length for MHCI). An antigen may also be a protein or other biomolecule that is presented on the surface of a cell, e.g. epidermal growth factor receptors or checkpoint proteins, which in the case of cancer cells are overexpressed therewith providing a differentiating feature when compared with non-diseased cells. Target cells may also include cells expressing auto-antigens, e.g. known to be involved in autoimmune diseases or cells infected with a pathogen, e.g. a virus.

It is understood that cellular avidity as used throughout herein comprises the overall strength of interactions occurring in a cell to cell contact, of an effector cell and a target cell, involving a diversity of molecules at the surfaces of the cells that interact. Such interactions may include a diversity of receptor-ligand pairs, among which e.g. a specific receptor-ligand interaction, occurring at the membrane surface of a cell. For example, when a T cell receptor of an effector cell triggers the formation of an immune synapse by recognizing an antigen presented by an MHC molecule at an antigen presenting cell, the synapse formation involves such multitude of interactions, as also other membrane bound molecules are involved in the interactions (such as integrins and the like). Hence, cellular avidity is not restricted to the interaction of e.g. the alpha and beta chain of the TCR (or of a CAR, or the like) and the antigen presented by MHC, but rather involves a multitude of interactions working jointly forming a strong bond between e.g. cells. It may also involve active signalling and processes internal to the cells such as e.g. during immune synapse formation. It is understood that the cellular avidity of an effector cell can be defined relative to its target cell.

In the means and methods in accordance with the invention, cellular avidity assessments are made by determining, e.g. quantifying, the effector cells that move away from target cells attached to a surface, when a force is applied on the effector cells away from the target cells. In the current invention, in particular, the force that is to be exerted is a centrifugation force. Since the formation of an immune synapse (and cellular avidity in general) is a dynamic process, the time period between establishing contact between an effector cell and a target cell and the subsequent application of a force is an important parameter that needs to be well controlled in order to achieve reproducible results.

In view of the above, it is understood that in the methods in accordance with the invention, the cellular avidity is assessed of an effector cell and a target cell. In the method, target cells are provided at a defined concentration, e.g. a cell suspension having substantially single cells in suspension. The target cells are optionally stained. It is understood that staining the target cells with a fluorescent marker may be highly advantageous in the methods of the invention as it allows to, using fluorescence microscopy, conveniently detect the target cells. It is understood that any suitable fluorescent staining may suffice, such as e.g. described in the example section. It is understood that a target cell may also e.g. constitutively express a fluorescent marker instead, e.g. GFP or the like. In any case, providing the target cells with a fluorescent marker can be contemplated in the methods in accordance with the invention.

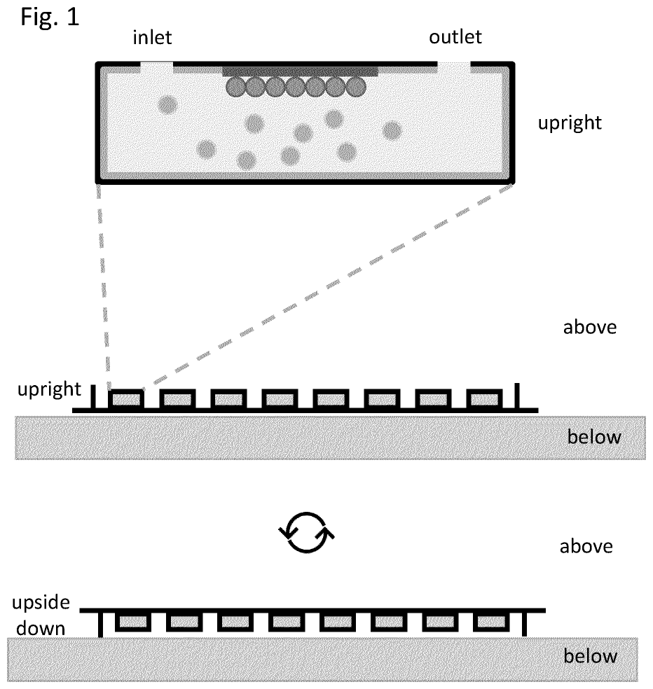

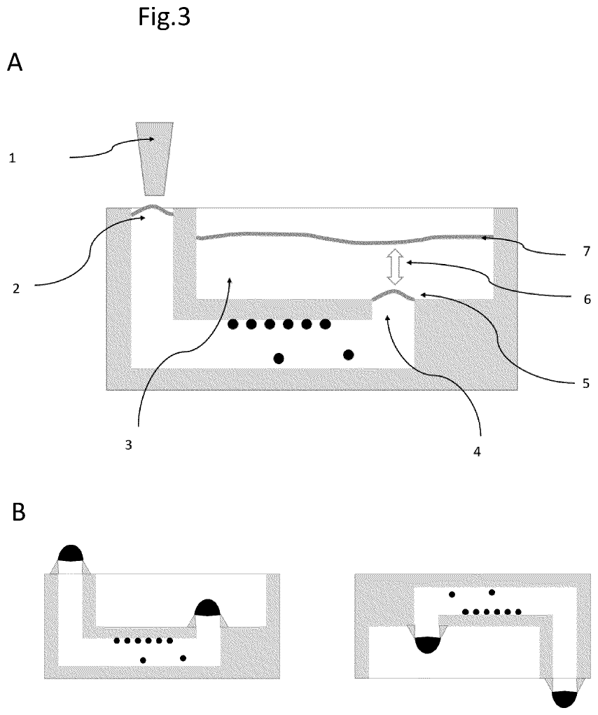

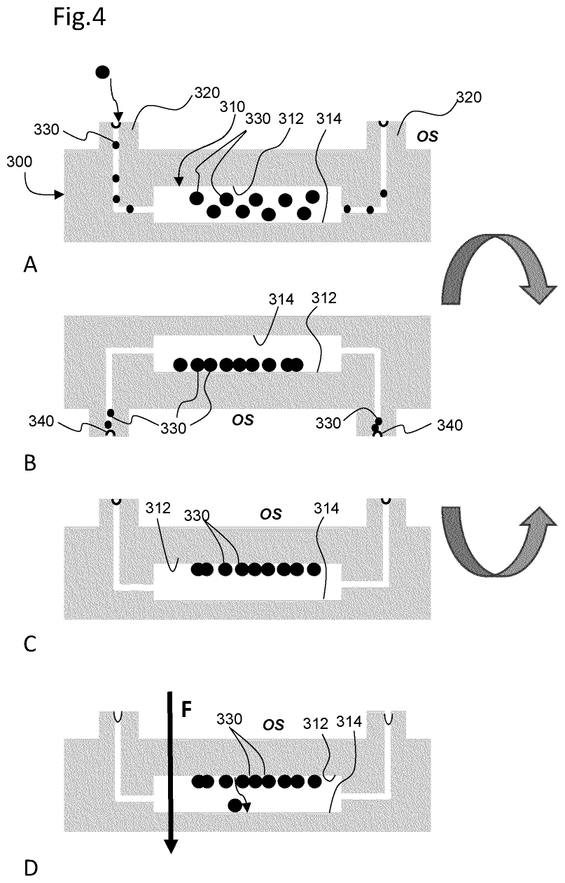

In addition, an avidity sample plate is provided with one or more wells with a ceiling surface coated for attachment of target cells, wherein at least the bottom surface is coated with anti-fouling, wherein the avidity sample plate is optionally provided with an identifier, and, optionally provided with one or more sensors. In addition, an avidity sample plate is provided with one or more wells with a ceiling surface coated for attachment of target cells, wherein optionally at least the bottom surface is coated with anti-fouling, wherein the avidity sample plate is optionally provided with an identifier, and, optionally provided with one or more sensors. In an embodiment, the avidity sample plate as used herein comprises a bottom surface that is coated with anti-fouling. In an embodiment, the avidity sample plate as used herein comprises a bottom surface that is made of material with anti-fouling properties. In an embodiment, the avidity sample plate as used herein comprises a bottom surface that is made of material that does not have anti-fouling properties and/or is not coated with anti-fouling. It is understood that identifiers and/or sensors can be removably attached to the avidity sample plate. As used herein, avidity sample plates are plates suitable for the means and methods in accordance with the invention, i.e. these are sample plates that are compatible with microscopy, comprise a surface to which target cells can attach, and allow for switching between upright and upside down position without letting attached target cells fall dry and which also allows exchange of fluids from the wells, e.g. of culture medium, cell suspension or washing fluids, or the like. As avidity sample plates are highly useful for cellular avidity measurements, one may also instead of the term avidity sample plate, refer to cellular avidity sample plate. A non-limiting example of a suitable avidity sample plate design includes plates with one or more wells, as depicted in FIG. 1, for example in a standard 96-wells format or the like, with in- and outlets compatible with multichannel pipetting devices or the like. Avidity sample plates are provided with one or more wells which may have a design as depicted e.g. in FIGS. 1, 3 and 4, e.g. with an inlet and outlet. In a preferred embodiment the height of a well or flow channel is from 200 to 400 μm. This height is optimal because the volume to area ratio is sufficient to allow for cells to create a monolayer and not run out of nutrients too fast. A height from 200 to 400 μm generally leads to a cell settling time of from 2.5 and 4.5 minutes. Increasing the height would mean increasing the cell settling time which is unwanted, as the higher the cell settling time is, the higher the heterogeneity in cell settling time becomes. For example, effector cells when flushed in will be distributed at multiple heights throughout the well or channel. Cells starting from a smaller height will take less time to sink down to the target cell monolayer and thus take less time to settle than cells starting from a larger height. This difference becomes larger, the larger the channel height.

As the wells on the plates may have inlets and outlets, and need to allow for inverting the plates without letting target cells attached to the ceiling surface fall dry, it is understood that inlets and outlets are designed such that the fluid remains in the wells during inverting steps, e.g. preferably because of surface tension forces or alternatively by closing inlets and outlets with a cap or plug. Alternatively to closing inlets and outlets with a cap or plug, the inlets and/or outlets may also be closed by other means such as a sealing membrane, film, foil or mat. In an embodiment the sealing membrane, film, foil or mat is pierceable, for example by a syringe, needle or pipette, without losing its sealing capability. In an embodiment liquids present in the inlets and/or outlets are removed.

It is understood that the avidity sample plates in accordance with the invention may have a ceiling surface, which is coated for attachment of target cells, and a bottom surface, which is at least coated with anti-fouling and/or at least has antifouling properties. It is understood that the avidity sample plates in accordance with the invention may have a ceiling surface, which is coated for attachment of target cells, and a bottom surface, which is not coated with anti-fouling or does not have antifouling properties. It is understood that bottom and ceiling are relative terms which depend on the starting position of the avidity sample plate.

Antifouling properties of a surface, such as of the bottom, may be achieved either by choosing a polymer that has inherent antifouling properties, or by applying a physiosorbed antifouling layer, e.g. a hydrophobic surface may be coated by applying an aqueous solution containing a PEG block co-polymer e.g. Pluronic-F127 that physiosorbes and presents a PEG-brush surface with low biofouling properties. Chemical functionalization may also be achieved by first attaching a chemically reactive linker to the surface, and then solution coating the device with an anti-fouling molecule that reacts with the activated linker molecule. Pattered coating may be achieved by using a photoactivatable linker and using a photo mask and UV illumination to activate the linker only in select regions of the device. In any case, suitable antifouling agents or antifouling properties of at least the bottom surface can be appropriately selected and applied in the avidity sample plates as described herein.

Suitable coating agents for attachment of target cells are known in the art. Such coatings preferably comprise a polypeptide. Polypeptides can be naturally occurring polypeptides and/or synthetic. Such a coating may comprise a polypeptide selected from the group consisting of fibronectin, poly-L-lysine, poly-D-lysine, poly-L-ornithine, laminin, collagen, fibronectin, fibrinogen, vitronectin, osteopontin thrombospondin, VEGF, VCAM-1, ICAM. In another further embodiment, the coating comprises one or more from the group consisting of fibronectin, poly-L-lysine, poly-D-lysine, poly-L-ornithine, laminin, collagen, fibronectin, fibrinogen, vitronectin, osteopontin thrombospondin, VEGF, VCAM-1, and ICAM. These polypeptides may attach to the surface and in their turn subsequently attach with the target cells thereby attaching the target cells to the surface. In any case, a suitable coating may be selected such that the target cells that are attached to the surface allow for the target cells to remain attached to the surface when applying a force on the effector cells that are bound thereto. In other words, when a centrifugal force is applied on the effector cells which allows for these cells to detach from the target cells, the target cells are to substantially remain attached to the surface.

The purpose of this differential coating is that in the upright position, target cells (or effector cells) do not attach to the bottom of the avidity sample plate because of the antifouling, and when in the upside down position, target cells can attach to the ceiling surface which is coated therefor, and effector cells can interact and bind to target cells attached to the ceiling surface. This allows for a well time-controlled method as interaction time between effector cells and target cells can be controlled and kept the same across the entire plate. Furthermore, as the avidity sample plate in accordance with the invention is to be compatible with centrifugation, it is understood that in case inlets and outlets are used, these are preferably to be positioned opposite to the bottom surface, such that when the plate is centrifuged to apply the force away from the target cells, the fluid contained in the wells is retained and there is no risk of leakage. Moreover, because of such positioning of in- and outlets, provided that the surface tension of the fluid contained in wells is sufficient to retain fluid during inverting the plates, it may not even be required to have plugs in the in- and outlets throughout the entire procedure, which is highly advantageous as it reduces the number of handling steps of the plates, allowing more convenient automation of (parts) of the process, e.g. with multichannel automated pipetting. Also, not using caps or plugs may avoid unwanted fluid flow induced when placing such caps or plugs on the fluid inlets and/or outlets. As indicated above, a suitable alternative for using caps or plugs on the inlets and/or outlets could be the use of other means such as a sealing membrane, film, foil or mat. In an embodiment the sealing membrane, film, foil or mat is pierceable, for example by a syringe, needle or pipette, without losing its sealing capability.

In case no caps or plugs are used, and inlets and outlets are used in avidity sample plate design, it may be preferred to rotate avidity sample plates, when switching between upside down and upright positions, along an axis of the plate which is selected to be parallel to the axis between inlets and outlets, inlets and outlets of all wells also being aligned on the plate. This way, unwanted fluid flow may be further minimized and/or avoided further during handling of the plates.

As said, the avidity sample plate is optionally provided with an identifier, and, optionally provided with one or more sensors as well. As the means and methods in accordance with the invention are aimed at an efficient and well controlled process, including an identifier, as well as one or more sensors, highly advantageously allows to control the steps of the methods as performed in accordance with the invention. Sequences of steps of the methods may be automated, and including an identifier, as well as one or more sensors, allows one to track avidity sample plates and the method steps with regard to set variables, as well as from a quality control perspective.

In any case, said avidity sample plate in accordance with the invention is provided. Next, the target cells are introduced, homogenized to one or more wells of the avidity sample plate, with the avidity sample plate in the upright position. It is understood that the target cells are homogenized prior to introduction of the target cells to one or more wells to ensure similar numbers of target cells are provided to each well. It is understood that the amount of cells provided is such to provide for a monolayer of target cells, i.e. the target cells are spread over the ceiling surface to provide for a single layer of target cells. It is understood that it is highly preferred to obtain a monolayer of target cells with a high confluency. Obtaining a high confluency may be achieved by introducing a number of target cells with which, once attached to the ceiling surface, high confluency is directly obtained. Conversely, one may also opt to introduce cells and allow target cells to grow until high confluency is obtained. A suitable confluency may be selected from at least 40%, at least 50%, at least 60%, at least 70%, at least 80%, at least 85%, at least 90%, at least 95%, or more.

Once the target cells are introduced, the avidity sample plate is positioned in an upside down position, thereby allowing the target cells to settle to the ceiling surface and attach thereto to form the monolayer. Once the target cells are attached to the ceiling surface, one may opt for culturing the target cells for some time, e.g. to reach high confluency as described above or to allow the cells to recover (e.g. in case target cells are introduced after trypsinization). The avidity sample plate may be held in the upside down position, but, it may also be opted to culture target cells in the upright position. Culturing in the upright position may be advantageous e.g. as risks of liquid leaks may be minimized, but the avidity sample plate may also be held in the upside down position. Accordingly, the next step, optionally, involves culturing the attached target cells, with the avidity sample plate in either upside down position or upright position, in a controlled incubator.

When the monolayer of target cells has been formed, after optional culturing, subsequently, the ceiling surface with attached target cells may be blocked with a blocking agent. Using a blocking agent may be useful in case the coating agent may induce unwanted aspecific binding of effector cells thereto. Suitable blocking agents include e.g. serum, such as FBS, BSA, which may be comprised in a suitable medium, and may also include polymers, e.g. pluronic-f68, or the like. Molecules with reactive linkers to the coating agent used may also be used as a blocking agent. For example, as coating agents in the current invention may comprise polypeptides with primary amine groups, agents that form a stable conjugate bond with primary amines may be used, i.e. NHS ester reagents, to effectively block coating agent remaining available. For example, a suitable NHS ester reagent may be a compound comprising at least one succinimidyl group, which functional group is capable of forming an ester bond, e.g. with a primary amine group on polypeptides.

In any case, as described above, an avidity sample plate is provided with target cells attached to the ceiling surface. Subsequently, the avidity sample plate is placed in an upright position. This in itself already exerts 1×g causing target cells that did not attach to the ceiling surface to settle down to the bottom of the wells. The avidity sample plate highly preferably is placed in a centrifuge in the upright position to subsequently spin down target cells that did not attach with at least 20×g. A higher force may be preferred, e.g. of 200×g, or more. The centrifugation step may also be performed at a force, acceleration, which is similar or exceeds the acceleration rate exerted on the effector cells in later centrifugation steps of the method for assessing cellular avidity. Hence, as said, subsequently, the avidity sample plate is placed in the upright position, optionally in a centrifuge and centrifuged to spin down target cells that did not attach to the ceiling surface.

Subsequently, the target cells attached to the ceiling surface may be washed with the avidity sample plate in the upright position to remove target cells that did not attach to the ceiling surface. It is understood that washing the cells includes at least exchanging the medium, e.g. for fresh medium. Washing the cells can also include exchanging for any fluid compatible with the cells, e.g. a PBS buffer or the like. Of course, after washing, wells are preferably provided with a suitable culture medium to ensure viability of the target cells. It is understood that this washing step may be optional in certain embodiments, for example, when the target cells provided all attach to the coated ceiling surface in which scenario there would be no need for a washing step.

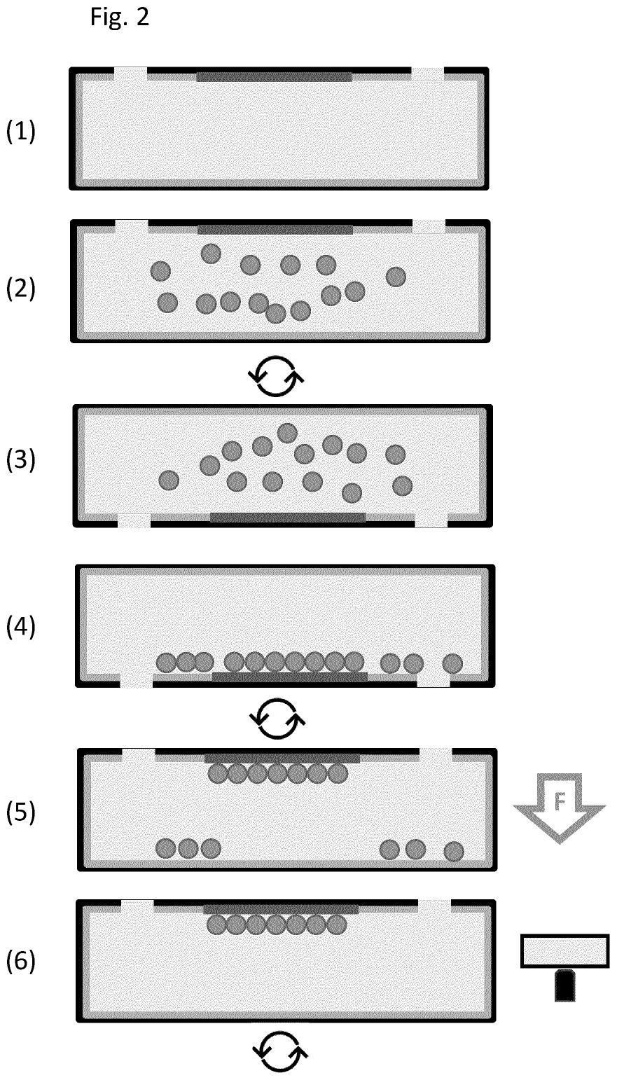

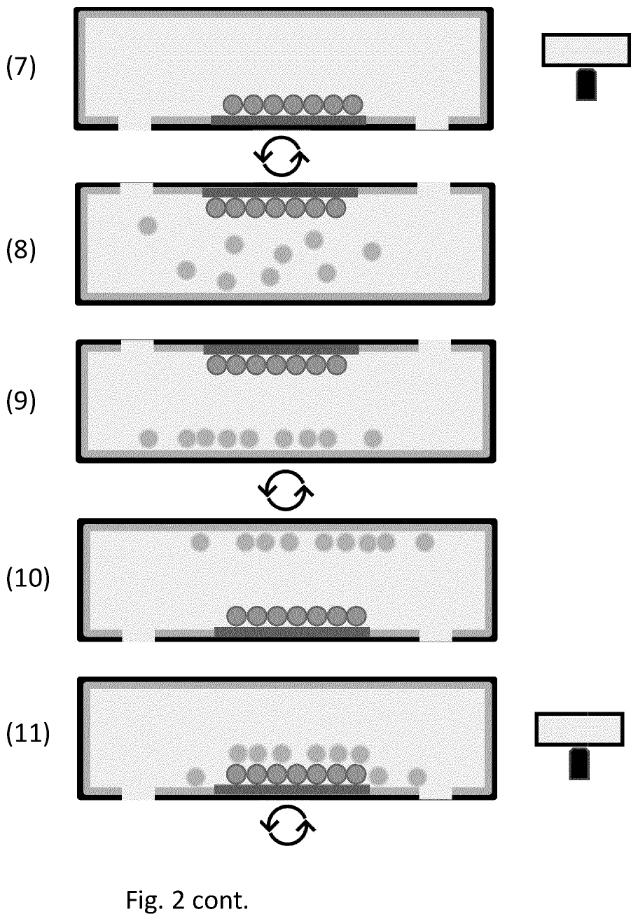

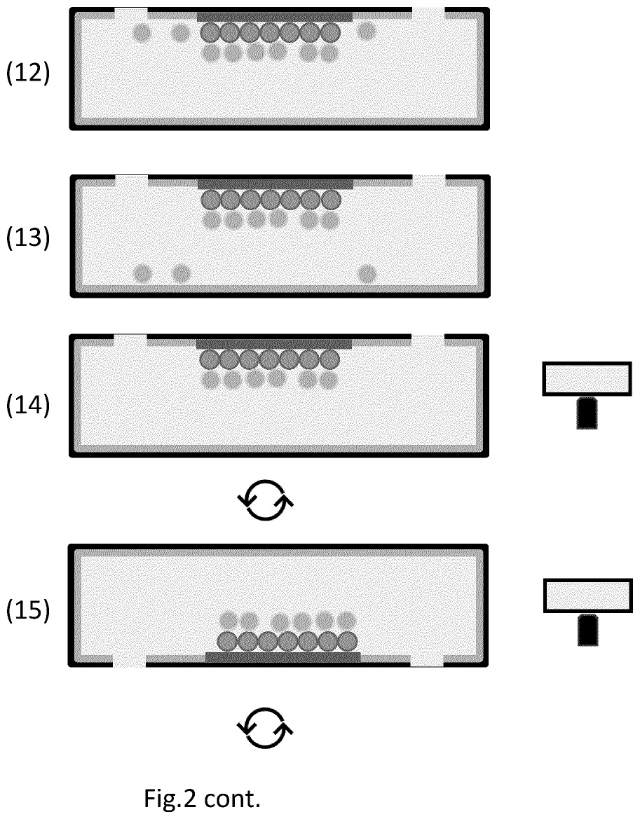

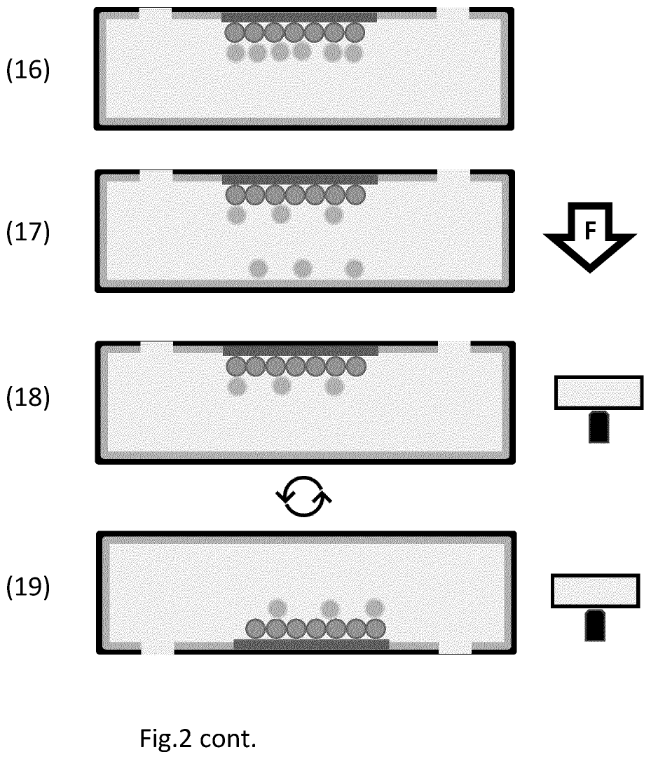

Next, optionally, an image is acquired of the monolayer of target cells attached to the ceiling surface. Acquiring an image at this stage is optional, but may be highly useful, for example in quality control, i.a. for checking the confluency and/or quality of the monolayer of target cells attached to the ceiling surface. Of course, the monolayer of target cells attached to the ceiling surface may also be imaged at a later stage. Having the target cells provided with a fluorescent label may be highly useful for acquiring an image. Of course, other dyes, e.g. non-fluorescent (absorptive) dyes may be contemplated instead as well. Acquiring an image is highly preferably performed using a microscope. As is understood a microscope may be positioned below the avidity sample plate, or above the avidity sample plate (see FIG. 1, with in FIG. 2 the microscope positioned at the below position). It is understood that plates can have protruding in- and outlets, which may interfere with providing a good perspective on the target cell monolayer. Hence, positioning of the microscope and/or avidity sample plate design may take into account such requirements. An image acquired includes an image taken at a single focal plane, e.g. utilizing fluorescence microscopy. It may also be contemplated to obtain multiple focal planes. Microscopy images can include multicolour and/or multifluorescent microscopy images taken at one or more focal planes. When utilizing a multicolour fluorescence microscope, focusing on effector cells, and target cell monolayers is more convenient and may not require multiple focal planes. Multiple focal planes, allows for focus stacking, also referred to as z-stacking to provide a composite image of the monolayer of target cells.

Once the target cells attached to the ceiling surface as a monolayer in the avidity sample plate is provided, subsequently, effector cells can be provided thereto. Hence, next, effector cells are provided at a defined concentration. The concentration may depend on many factors including the height of the well or flow channel used, for example the concentration of effector cells may be 0.5-1e6/ml for a height of 400 μm and 1-2e6/ml for a height of 200 μm. The indicated concentrations are optimal for the indicated heights as it provides cell numbers that enable to distinguish single cells with imaging software used in the art and at the same time to have good data quality.

Optionally, and preferably, the effector cells are stained, e.g. with a fluorescent marker. As said, having cells fluorescently labelled, e.g. as described in the example section, or through e.g. stable transduction with GFP or the like, allows for convenient detection of effector cells utilizing fluorescence microscopy. The effector cells are introduced, after homogenization, to one or more wells of the avidity sample plate in the upright position and the effector cells are allowed to settle to the bottom of the avidity sample plate. In this step, the avidity sample plate is in the upright position such that the effector cells do not yet substantially interact with the target cells attached to the ceiling surface. By allowing the effector cells to settle to the bottom of the avidity sample plate, all effector cells have the same starting position and distance to the ceiling surface. In an embodiment the step of allowing the effector cells to settle to the bottom of the avidity sample plate is optional. Next, the avidity sample plate is placed in the upside down position, and the effector cells are allowed to settle onto the monolayer of target cells attached to the ceiling of the avidity sample plate and the effector cells are allowed to incubate on the target cells, for a defined time. Incubation may be performed in a controlled incubator. By performing the method this way, highly advantageously, the duration of placing the avidity sample plate in the upside down position, and subsequent centrifugation, to the end of the incubation is the same for all wells of the avidity sample plate and can be well controlled.

It is understood that in the methods in accordance with the invention or the like, from placing the avidity sample plate, having the effector cells introduced and optionally settled to the bottom of the avidity sample plate, in the upside down position up until centrifugation, the steps of the methods are highly preferably well controlled, e.g. in order to allow to compare different cellular avidity assessments made with different avidity sample plates. It is understood that the duration of the step of allowing the effector cells to settle on the target cell monolayer, and the duration of time after the avidity sample plate is placed in the upright position (during which e.g. an image may captured, and the plate needs to be placed in the centrifuge), is preferably well controlled, and may be largely determined by the time procedural steps require. The time of incubation of the effector cells that settled on the target cells, may be selected from the range of 1 second to 4 hours, from 1 second to 3 hours, from 1 second to 1 hour, from 1 second to 40 minutes, from 1 second to 30 minutes, from 1 second to 25 minutes, from 1 second to 20 minutes, from 1 second to 15 minutes, from 1 second to 10 minutes. This way, and highly preferred, the total time from placing the avidity sample plate, having the effector cells introduced and optionally settled to the bottom of the avidity sample plate, in the upside down position up until centrifugation, is well controlled and can be reproduced between cellular avidity assessments.

Optionally, and highly preferably, an image is captured, with the avidity sample plate in the upside down position, of the effector cells and the monolayer of target cells. Capturing an image at the end of the incubation time is preferred, as this allows one to define e.g. in a defined field of view of the target cells attached to the ceiling surface effector cells that are in contact with the target cells. This includes effector cells bound to target cells and also effector cells not bound to target cells. It may also be contemplated to capture multiple images, or even a video, during the defined time, or part thereof. In any case, capturing an image at the end of incubation may be preferred, e.g. capturing a brightfield image, and fluorescent colours as well. For example, a multicolour image may be captured, e.g. of target cells and effector cells which are differently labelled. It may be contemplated to determine images from multiple focal planes and perform z-stacking to provide a more detailed or contrast enhanced image. As for determining a cellular avidity score, the number of effector cells in a defined field of view, e.g. of the ceiling surface with target cells attached thereto, is of importance, it may also be contemplated to calculate the number of effector cells that theoretically should be present in a defined field of view when one knows the amount of effector cells provided in each well. Hence, capturing an image is optional, but nevertheless highly preferred as it allows to more accurately control e.g. the measurement of cellular avidity.

Next, the avidity sample plate is placed in the upright position. In the upright position, gravity force is exerted on the effector cells of about 1×g, which means that effector cells that are unbound or did not substantially bind to target cells can settle down to the bottom of the avidity sample plate. Hence, optionally the effector cells that did not bind to the target cells are allowed to settle to the bottom of the avidity sample plate, and optionally, said effector cells that settled to the bottom are collected or washed away. As it may be of interest to collect fractions of effector cells, one may contemplate to collect cells that did not bind to target cells. Alternatively, one can just simply remove these effector cells by washing them away. Now, subsequently, like above, optionally, an image is captured of the effector cells bound to the monolayer of target cells, with the avidity sample plate in the upright position. This image is effectively the same as the picture taken above in the upside down position, the difference being that unbound effector cells are not present in the captured image. Hence, instead of the image above, one may equally well capture this image instead.

Next, the avidity sample plate is placed in the upright position in the centrifuge and, at a defined centrifugal force, e.g. of 1000×g, centrifuged for a defined time. In this step, a force is exerted on the effector cells that are bound to the target cells, away from the ceiling surface to which the target cells are attached. When the strength of binding between the effector cells and target cells is lower as compared with the centrifugation force, effector cells can become unbound and move away from the ceiling surface to the bottom of the well. After this step, subsequently, an image is captured of the effector cells bound to the monolayer of target cells, with the avidity sample plate in the upright position. The avidity sample plate is kept in the upright position to avoid repeated interaction with the target cells attached to the ceiling surface. Optionally, the effector cells that become unbound in this step may be collected or washed away, before or after capturing the image.

The above steps of centrifugation, capturing an image, and optionally washing away or collecting effector cells that became unbound may be repeated one or more times, with for each consecutive time, an increasing centrifugal force. In an alternative embodiment, centrifugation may be repeated with the same centrifugal force, increasing the time with each consecutive centrifugation the effector cells are subjected to the centrifugal force. This way, e.g. a cellular avidity plot or curve can be determined, e.g. plotting cumulatively the number of cells that became unbound, or different fractions of cells can be collected. Finally, the effector cells that remain bound to the target cells after the image was captured, may be collected. It is understood that of course this collection step is to be performed after the last imaging step in case the steps of centrifugation, capturing an image, and optionally washing away or collecting, were repeated one or more times. Collecting these cells may be of interest as these cells were capable of forming the strongest bond between effector cells and target cells and/or were capable of forming a strong bond during the selected incubation time.

With regard to capturing images, it is understood that images may be captured from below or from above the avidity sample plate, wherein the avidity sample plate may be in the upright or upside down position (see i.a. FIG. 1). In FIG. 2, it is schematically depicted to have the microscope positioned below the avidity sample plate. For example, of target cells attached to the ceiling surface an image may be collected with a microscope having the avidity sample plate either in the upright position, or in the upside down position. The working distance between the microscope and the effector cells/target cells attached to the ceiling surface is larger in the upright position as compared with upside down position, in the scenario wherein the microscope is positioned below the avidity sample plate. In case it is contemplated to collect images in upright and upside down positions of the avidity sample plate it is highly preferable to have the microscope equipped with multiple objectives to accommodate acquiring images in these different positions. When collecting images in the upside down configuration (e.g. when the objective images through the ceiling instead of through the bottom), it may be possible to use an objective with a shorter working distance and therefore a higher numerical aperture and/or a larger magnification. This may be advantageous to obtain more detailed images of cells containing more information (e.g. when determining markers as described below). It may be preferred to have the microscope in the below position, e.g. in an avidity measuring unit as provided herein, as it allows for capturing images from below, while allowing for access from above to the avidity sample plate, e.g. with a (multichannel and/or robotic) liquid handling system. Hence, in a preferred embodiment, images may be captured using a microscope from below the avidity sample plate. In another embodiment, images are captured using a microscope from above of the avidity sample plate. Highly preferably, the said microscope comprises multiple fluorescence channels, e.g. two, three, four or more fluorescence channels. Such is highly preferred as it allows for performing multiplexed fluorescence microscopy enabling to increase the number of samples per avidity sample plate.

It is understood that the avidity sample plate preferably is made from or comprises a material which allows for microscopy as envisioned herein. Suitable materials are known in the art, such as polymers such as polystyrene, which can accommodate microscopy. It is highly preferred to have the avidity sample plate comprise or consist of a cyclic olefin copolymer (COC) or cyclic olefin polymer (COP), the latter which uses a single type of monomer, which is highly useful for microscopy applications while at the same time highly suitable for accommodating cells, e.g. for cell culture. Hence, in a further embodiment, the avidity sample plate is made from or comprises a polymer, such as COC or COP. Preferably, the avidity sample plate is in a standardized multi-well format, e.g. a 48-well, a 96-well, or 384-well format. Such formats allow for easy integration into high-throughput formats and devices, e.g. utilizing multichannel liquid handling systems, and the like.

In another embodiment, in the methods in accordance with the invention the ratio of the number of target cells to the number of effector cells is in the range of 1:1 to 100:1, preferably about 10:1. It is understood that in a well wherein the target cells are to be attached to the ceiling surface, the number of target cells preferably is equal or larger than the number of effector cells. This way, an effector cell always will have contact with a target cell during incubation, at least if the monolayer of target cells has high confluency. Hence, in a first embodiment, a ratio of at least 1:1 is selected. It may be advantageous to select an upper limit of about 100:1, such that the field of view may be selected which is not too large and a sufficient number of effector cells can be assessed, e.g. when counting the number of effector cells. Preferably, a ratio of about 10:1 of the number of target cells to the number of effector cells is selected, as such a ratio allows for on average a surface area representing about 10 target cells to be available for each effector cell.

It is understood that as the avidity sample plate can comprise a large number of wells, each well may be provided with a different effector cell type, or a different effector cell clone. For example, with regard to different effector cell types, these may be effector cells obtained from different donors, e.g. when a defined CAR is provided to effector cells isolated from the PBMCs obtained from different donors. Furthermore, from the same donor, different effector cell types, or cell populations, may be provided, e.g. by providing T cells, and NK cells from the same donor, wherein T cells of said donor may be further differentiated as T-helper cells (CD4+/CD3+) and cytotoxic T cells (CD8+/CD3+). Furthermore, effector cells may also be provided with different receptors, such as different CAR receptors. Effector cells carrying the same CAR receptor may be defined as representing or being representative of an effector cell clone, although it is understood and accepted that the effector cells in which the same CAR receptor has been introduced may not necessarily be identical with regard to phenotype or even genotype. Accordingly, in another embodiment, multiple effector cell types or multiple effector cell clones are provided, wherein each of the effector cell types or clones is provided to a different well.

As the current invention allows for fluorescence multiplexing, it is not required to provide each effector cell clone or each effector cell type to a different well. Highly advantageously, in a further embodiment, wherein multiple effector cell types and/or multiple effector cell clones are provided, each of the effector cell types or each of the effector cell clones is provided with a different staining, and wherein multiple effector cell types or clones are combined and provided to individual wells. For example, in case one wishes to perform experiments in triplicate, and using 4 different fluorescence stainings, one can combine 4 different effector cell clones and/or types per well. In an avidity sample plate with a 96-wells format, this would mean that (96/3)×4=124 different effector cell clones and/or types can be analysed on one plate. Because multiple different cell clones and/or types are used in a single well and across an avidity sample plate, cellular avidity assessment can also be better validated across the plate, because each well is controlled by having multiple samples, and because each unique sample is provided across the plate.

Also, in case images are taken at different time points and/or after repeated centrifugation steps, having cells with different labels also allows for improved tracking of individual cells in an image. It is understood that in case tracking of cells is of importance, this is likewise advantageous for effector cells that are identical or highly similar, e.g. as having the same phenotype and carrying the same receptor.

Further complexity may be added to the analysis allowing even more different effector cells per cell when cells are allowed to have for example double staining. With double staining with red, blue, yellow and green (r, b, y, g) ten unique combinations can be made (g, b, y, r, g/b, g/y, g/r, b/y, b/r, y/r). Hence, already with four different fluorescence channels in microscopy, a large variety of experimental conditions involving i.a. different effector genotype and phenotypes, donor types, and/or different effector cell clones may be highly efficiently analysed.

Furthermore, control effector cell clones may be included in each well, or a selection of wells, as well to provide for control across the entire plate. Control effector cell clones include positive effector cell controls, i.e. having a high cellular avidity with the target cells, or negative effector cell controls, i.e. having a low or no detectable cellular avidity with the target cells. One may even contemplate to combine both positive and negative effector cell controls in a well, e.g. a substantial portion of the wells of the avidity sample plate, or even all of the wells. An avidity sample plate run may include such controls and when the values obtained in a cellular avidity assessment comply with defined reference ranges, such an avidity sample plate run may be regarded to be validated. Hence, in one embodiment, in addition to the effector cells, which may be multiple effector cell types and/or multiple effector cell clones, furthermore, control effector cells are provided as well. In a further embodiment, said control effector cells are provided to separate wells. In yet another further embodiment, said control effector cells are included in one or more wells, or in each well, wherein the control effector cells are differently stained as compared with the effector cells. In still another further embodiment, the ratio of the number of control effector cells to the number of effector cells in a well is in the range of 1:1 to 1:10.

It is understood that the target cells may be stained as well, e.g. with a fluorescence marker, or the like. Such staining is highly preferably different from the staining of the effector cells such that in images effector cells that did not bind to target cells but e.g. inadvertently bound to the ceiling surface directly instead because of e.g. the absence of target cells at certain areas within the monolayer of target cells at the ceiling surface. Hence, areas within the field of view of an image wherein no target cells are detected may not be taken into account when assessing cellular avidity between effector cells and target cells. It may also be contemplated to provide for different target cells, e.g. providing target cells originating from different donors each donor presenting the same antigen. Such different target cells may include control target cells, for example target cells that do not express a selected antigen and/or do highly efficiently express/present a selected antigen. More preferably, the different target cells are provided in separate wells and not mixed. Nevertheless, it may be contemplated to mix different target cells, e.g. by providing a panel of different target cells in a well. For example, one may include control target cells, to which effector cells are not to bind, along with target cells. In methods in accordance with the invention, such may allow to select effector cells that do bind with the target cells, but at the same time do not bind with the control target cell. Accordingly, in a further embodiment, the target cells are stained as well, which staining is different from the staining of the effector cells. In yet another embodiment, wherein different target cells are provided, optionally including control target cells, said different target cells are provided to separate wells of the avidity sample plate. In still another embodiment, wherein different target cells are provided, optionally including control target cells, said different target cells are provided and combined in wells of the avidity sample plate.

In yet another embodiment, the images are analysed in the assessment of cellular avidity between an effector cell and a target cell. It is understood that such an analysis may be performed at the site where the avidity sample plate is subjected to the method of the invention, e.g. by capturing the images and processing and assessing the images with analysis software. It is understood that such an analysis may also be performed at a site remote from where the avidity sample plate is subjected to the method of the invention.

Image analysis includes analysing the one or more captured images with regard to the number of effector cells in the captured image, e.g. in defined field of view. It is understood when of a well, multiple images are captured, the same defined field of view is used in the analysis. Based on the captured images, a cellular avidity score can be determined. For example, when the ratio is calculated of the number of effector cells that remain after centrifugation to the number of effector cells that initially interacted with the target cells, e.g. in a field of view, a cellular avidity score is provided. Of course, it may not be necessary to always calculate a ratio relative to the number of effector cells initially provided, determined either by calculation or by analysis of an image, e.g. of a defined field of view. One may also, calculate a score relative to e.g. a reference value or to the numbers determined of effector control cells, which, likewise provides for relevant information related to cellular avidity between effector cells and control cells. Accordingly, in further methods in accordance with the invention, analysing the captured images comprises determining the number of effector cells in captured images. In yet another embodiment, said determined number of effector cells are used to calculate a cellular avidity score, e.g. for each well and/or for each effector cell type and/or effector cell clone comprised in each well, if multiple effector cell types and/or effector cell clones are introduced in wells as described above. In one embodiment, the cellular avidity score is the ratio of the number of effector cells that remained bound to the number of effector cells initially provided. In another embodiment, the cellular avidity score is the ratio of the number of effector cells that remained bound after centrifugation, to the number of effector cells initially provided. In yet another embodiment, the cellular avidity score is the ratio of the number of effector cells that remained bound after centrifugation, to the number of effector control cells that remained bound. In any case, different cellular avidity scores may be calculated and contemplated which take into account the number of effector cells that remained bound after the centrifugation step. As long as the cellular avidity score can provide for a useful relative parameter which allows to rank or relate different cellular avidity scores determined for different effector cell target cell combinations, such a cellular avidity score can be contemplated.

It is understood that a synapse is a specialized structure that forms when the plasma membranes of two cells come into close proximity to transmit signals. Synapses can form between cells expressing a receptor and target cells, when e.g. the cells with the receptor are effector cells. Cells of the immune system form synapses that are essential for cell activation and function. Lymphocytes such as T cells, B cells and natural killer (NK) cells form synapses that can be referred to as immunological synapses. Such a synapse typically forms between effector cells and target cells, e.g. cells presenting an antigen. A non-limiting example is e.g. a T cell or a CAR-T cell and a cancer cell. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the case of a T cell, a synapse can be formed between the lymphocyte and antigen-presenting cells (APCs) during the recognition of the peptide antigen-major histocompatibility complex (pMHC) ligand by the T cell antigen receptor (TCR). The TCR and pMHC are both membrane-bound so the TCR will only be triggered by its ligand at the interface between T cells and APCs. A synapse can be observed at the T cell-APC interface as concentric rings by confocal microscopy, often referred to as “bull's eye” (Huppa, J. B., & Davis, M. M. (2003). T cell-antigen recognition and the immunological synapse. Nature Reviews Immunology, 3 (12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2) CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2) CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 Z-chain. Later generations combined intracellular signalling domains from various costimulatory protein receptors (e.g., CD28, 41BB, ICOS) incorporated in the cytoplasmic tail of the CAR to enhance signalling further. Further generations also incorporated in their design an inducible release of transgenic immune modifiers, such as IL-12, to shape the tumour environment by augmenting e.g. T cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized pattern. Multiple CAR microclusters form and signalling molecules, which are dispersed in the centre of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved e.g. in T cell activation and/or synapse formation.

In any case, in the means and methods in accordance with the invention, one or more markers associated with synapse formation can be determined as well. Thus, in a further embodiment, the marker associated with synapse formation is determined in either the target cell or the effector cell. In another embodiment, in the methods in accordance with the invention one or more markers associated with synapse formation are determined and the one or more markers are determined in the effector cells and/or in the target cells.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse in which, as described above but not necessarily limited thereto, markers associated with synapse formation are associated.

In a further embodiment, the marker for synapse formation is calcium signalling, which can be detected with fluorescent calcium indicators, such as Fura2 AM (available from Invitrogen, item nr. F1221), which marker is suitable for detection in effector cells and in live cells. Other suitable dyes to detect calcium signalling can be selected from the group of Fura Red AM, Indo-1, pentapotassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, pentapotassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used a calcium signalling indicators, such as the Invitrogen FluoVolt™ Membrane Potential Kit (Catalog number: F10488). Depolarization of the synapse forming cells by using a slow-response potential-sensitive probe such as Invitrogen DiSBAC2 (3) (Bis-(1,3-Diethylthiobarbituric Acid)Trimethine Oxonol), Catalog number: B413. Hence, in another embodiment the marker for synapse formation is detected utilizing membrane potential dyes.

In another embodiment, the marker for synapse formation is cytoskeleton rearrangement, which can be detected in effector cells with live or fixed cells, using cell staining, e.g. F-actin can be detected with Phalloidin conjugates or CellMask™ (Invitrogen, item nr A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is detected with a stain selected from the group consisting of Phalloidin conjugates, CellMask™, SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation effector cells will stop moving and remain into contact with the target cell with which it forms a synapse. Cell motility can be detected by membrane staining of effector cells and imaging, or using brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cell for which the marker associated with synapse formation was detected can easily be tracked.

As described above, in accordance with the invention, markers associated with synapse formation can be determined, e.g. via utilizing labels or the like. Accordingly, in a further embodiment, the means and methods also provide for detecting immune synapses as well, for which fluorescent labels are highly useful as well. When analysing the captured images, cellular avidity scores can be calculated based on the number of effector cells that formed a synapse with the target cells. Hence, in another embodiment, immune synapses are detected. Such detection preferably takes place with captured images. In a further embodiment, analysing the captured images comprises determining the number of effector cells that formed a synapse. In yet another further embodiment, the determined number of effector cells that formed a synapse is used to determine a cellular avidity score.

It is understood that detection of synapses and/or further markers may also be performed on the effector cells that remain bound to the target cells. Cells may be fixed or live. In such a scenario, it may also be envisioned to detect synapses not in images obtained from the avidity sample plates. Effector cells that remained bound to the target cells may be collected from the avidity sample plates. Hence, in one embodiment, the method of the invention comprises a further collection step, wherein the effector cells that remain bound to the target cells are collected, which comprises subjecting the effector cells bound to the attached target cells to a trypsin treatment and/or resuspension step.

When utilizing avidity sample plates in the methods of the invention, in order to monitor the steps of the methods, the duration of one or more steps can be recorded. All durations of the steps of the methods may be recorded. However, it is in particular of importance to monitor the time of placing the avidity sample plate in the upside down position to allow the effector cells to settle on the target cells attached to the ceiling surface of the avidity sample plate and subsequent incubation, for a defined time, until placing the avidity sample plate in an upright position (e.g. steps o) to q) of a method as described herein i.a. above). Moreover, it is highly preferred to control the duration of each step of the method. This may be done manually, but highly advantageously, this can be done with an automated process, e.g. using robotics or the like to handle the avidity sample plate, which allows for good control of switching the plate between upright and upside down positions. It is understood that not all handling may be performed with an automated process, some steps of the method may be performed manually, while other steps of the method may be performed automatically. Hence, in one embodiment, the duration of one or more steps, for example of each step, is recorded. In a further embodiment, the duration of one or more steps is controlled, preferably by an automated process. In still a further embodiment, the duration of each step is controlled. It is understood that recording the duration of the steps of the methods may serve as a means to control the method or to validate the method.

As described herein, in the methods in accordance with the invention the avidity sample plate may be further provided with one or more sensors. Providing the avidity sample plate with one or more sensors further allows to monitor and/or validate the steps of the method, e.g. when carried out (in part) as an automated process. In particular, the one or more sensors may be an accelerometer, which allows to monitor the orientation of the plate and the forces exerted, e.g. during changing the position of the avidity sample plate between an upright and upside down position, and also to monitor e.g. the forces exerted on the effector cells during the one or more centrifugation steps. Hence, having an accelerometer as a sensor in the plate is highly useful. Further useful sensors the avidity sample plate may be provided with is e.g. a thermometer, a humidity sensor, and/or a CO₂ sensor, which sensors may be useful to ensure that cell culture conditions the avidity sample plate is held in compliance with defined parameter ranges, further controlling the entire process. It is understood that highly preferably, sensor readings are registered in time. Hence, in one embodiment, the avidity sample plate is provided with one or more sensors and the one or more sensors is an accelerometer, and wherein in the method sensor readings are registered in time. In another embodiment, the avidity sample plate is provided with one or more sensors and the one or more sensors are selected from an accelerometer, a thermometer, a humidity sensor, a CO₂ sensor, and wherein in the method sensor readings are registered in time. It is understood that the sensor readings, like the captured images, may be used in the subsequent analysis and/or validation of a cellular avidity assessment. As said, it is understood that such an analysis may be performed at the site where the avidity sample plate is subjected to the method of the invention. For example, such analysis can include capturing the images, which can include sensor readings as well, and processing and assessing the images and further data provided with analysis software. It is understood that such an analysis may also be performed at a site remote from where the avidity sample plate is subjected to the methods of the invention.

As described above, the methods of the invention are highly useful to determine the cellular avidity between an effector cell and a target cell. As it is understood, additional molecules may also be included in the methods of the invention, as these may also affect the cellular avidity between an effector cell and a target cell. In particular, cell engagers may be of interest. Cell engagers include antibodies, or the like, which are capable of binding to a target cell and a cell of interest, e.g. an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, which may include synapse formation, as induced by a cell engager between a target cell and an effector cell. Hence, accordingly, in the means and methods in accordance with the invention, a cell engager may be provided in addition, and e.g. the cellular avidity score is determined, induced by said cell engager between a cell of interest (e.g. an effector cell) and a target cell, said cell engager having a binding region capable of binding the cell of interest (e.g. an effector cell) and a binding region capable of binding the target cell. It is understood that the incubation step in the methods of the invention can thus be performed in the presence of the cell engager to allow the cells to interact, i.e. effector cells and target cells, and form a bond, e.g. a synapse, via the cell engager.

Accordingly, in one embodiment, in methods in accordance with the invention, a cell engager is provided capable of binding an effector cell and the target cell and inducing synapse formation, and the cell engager is included in the incubation step. Furthermore, such methods may be highly useful for screening cell engagers, e.g. by identifying cell engagers that are particular capable of inducing e.g. synapse formation.

Of course, the methods of the invention are not limited to the use of cell engagers, and other molecules, or combinations thereof, that may affect the interaction between effector cell and target cell may be used therein. Hence, in a further embodiment, additional molecules (e.g. cell engagers) and/or compounds are added before, during or after introducing the effector cells in the avidity sample plate.

Also provided in accordance with the invention is an avidity measuring system, to which also may be referred to as a cellular avidity measuring system. In one embodiment, such an avidity measuring system comprises a centrifuge unit, an avidity measuring unit, an avidity sample plate holder, and one or more avidity sample plates that fit in the avidity sample plate holder. In another embodiment, the avidity measuring system further comprises an incubator unit. It is understood that the centrifuge unit and/or avidity measuring unit may have climate control like incubators. However, in case multiple avidity sample plates need to be processed, it may be advantageous to have a separate incubator unit, in particular in case e.g. incubation steps and/or culturing steps are selected which take a substantial amount of time.

The said avidity sample holder is designed such that it is compatible with the centrifuge unit, the avidity measuring unit, and, optionally, the incubator unit. In yet another further embodiment, the avidity measuring unit comprises a microscope and a liquid handling unit. It is understood that the avidity measuring unit may also be provided with means that allows to switch the orientation of the avidity sample plates between upright and upside down position, e.g. through robotics or the like. Furthermore, the microscope of the avidity measuring unit is in a further embodiment equipped with a fluorescence microscope having one more fluorescent channels.

In yet another embodiment, an avidity measuring unit is provided, compatible with the avidity sample plate, wherein the avidity measuring unit comprises a microscope and a liquid handling unit. It is understood that the avidity measuring unit may also be provided with means that allows to switch the orientation of the avidity sample plates between upright and upside down position, e.g. through robotics or the like.

Furthermore, the microscope of the avidity measuring unit is in a further embodiment equipped with a fluorescence microscope having one more fluorescent channels.

The avidity measuring units, and avidity measurement systems as described herein are in particular suitable to perform the methods of the invention.

Instead of preparing an avidity sample plate as described above with target cells attached thereto, using the steps as described herein, one may also provide a sample plate comprising a monolayer of target cells. Hence, in an another embodiment, a method is provided of assessing cellular avidity of an effector cell and a target cell comprising the steps of:
a) providing an avidity sample plate with one or more wells with a ceiling surface comprising a monolayer of target cells, wherein at least the bottom surface is coated with anti-fouling, wherein the avidity sample plate is optionally provided with an identifier, and, optionally provided with one or more sensors;
   wherein said target cells are optionally stained
b) providing effector cells at a defined concentration;
c) optionally, and preferably, staining the effector cells;
d) introducing the effector cells, homogenized, to one or more wells of the avidity sample plate in the upright position and optionally allowing the effector cells to settle to the bottom of the avidity sample plate;
e) placing the avidity sample plate in the upside down position to allow the effector cells to settle on the monolayer of target cells attached to the ceiling of the avidity sample plate and incubating the effector cells with the target cells, for a defined time;
f) optionally, capturing, with the avidity sample plate in the upside down position, an image of the effector cells and the monolayer of target cells;
g) placing the avidity sample plate in the upright position;
h) optionally, allowing the effector cells that did not bind to the target cells to settle to the bottom of the avidity sample plate, and optionally, collecting or washing away said effector cells that settled to the bottom;
i) optionally, capturing an image of the effector cells bound to the monolayer of target cells, with the avidity sample plate in the upright position;
j) placing the avidity sample plate in the upright position in the centrifuge and centrifuging, at a defined centrifugal force, e.g. 1000×g, for a defined time;
k) capturing an image of the effector cells bound to the monolayer of target cells, with the avidity sample plate in the upright position;
l) optionally, collecting or washing away effector cells that become unbound from the target cells, before or after capturing the image;
m) optionally, repeating steps j) to l), one or more times, with for each consecutive time an increasing centrifugal force;
n) optionally, collecting the effector cells that remain bound to the target cells after the image was captured.

In an embodiment of the above-mentioned method the coating of the bottom surface with anti-fouling is optional. In an embodiment of the above-mentioned method the bottom surface is made of a material with anti-fouling properties. In an embodiment of the above-mentioned method the bottom surface does not comprise a coating with anti-fouling and/or is not made of a material with anti-fouling properties. In another embodiment of the above-mentioned method in step (b) the cells provided at a defined concentration are tumour cells such as for example tumour B cells.

It is understood that instead of having the ceiling surface coated with target cells, the ceiling surface may also be provided with any other suitable surface with which one has interest of studying the interaction with, of e.g. with effector cells or any other cell of interest. Hence, in another embodiment, instead of providing target cells attached on the ceiling surface, a functionalized wall is instead provided which provides a suitable surface to study the interaction with of effector cells in accordance with the invention as described herein throughout. A functionalized wall in accordance with the invention may present ligands/receptors in a similar fashion as they are presented on a cell surface, e.g. in a lipid bilayer, or the like. A functionalized wall in one embodiment thus preferably may be functionally equivalent to target cells attached to a surface, and may mimic target cells attached to a surface. A functionalized wall in accordance with the invention may also present polypeptides, proteins, binding molecules (e.g. antibodies or parts or derivatives thereof), peptides, antigens, ligands, receptors, compounds, to name just a few. Such a functionalized wall provides a suitable surface to study the interaction with cells or other objects in accordance with the invention as described herein throughout.

Hence, in an another embodiment, a method is provided of assessing cellular avidity of an effector cell with a functionalized wall comprising the steps of:
a) providing an avidity sample plate with one or more wells with a ceiling surface comprising a functionalized wall, wherein at least the bottom surface is coated with anti-fouling, wherein the avidity sample plate is optionally provided with an identifier, and, optionally provided with one or more sensors;
b) providing effector cells at a defined concentration;
c) optionally, and preferably, staining the effector cells;
d) introducing the effector cells, homogenized, to one or more wells of the avidity sample plate in the upright position and optionally allowing the effector cells to settle to the bottom of the avidity sample plate;
e) placing the avidity sample plate in the upside down position to allow the effector cells to settle on the functionalized wall of the avidity sample plate and incubating the effector cells with the functionalized wall, for a defined time;
f) optionally, capturing, with the avidity sample plate in the upside down position, an image of the effector cells;
g) placing the avidity sample plate in the upright position;
h) optionally, allowing the effector cells that did not bind to the functionalized wall to settle to the bottom of the avidity sample plate, and optionally, collecting or washing away said effector cells that settled to the bottom;
i) optionally, capturing an image of the effector cells bound to the functionalized wall, with the avidity sample plate in the upright position;
j) placing the avidity sample plate in the upright position in the centrifuge and centrifuging, at a defined centrifugal force, e.g. 1000×g, for a defined time;
k) capturing an image of the effector cells bound to the functionalized wall, with the avidity sample plate in the upright position;
l) optionally, collecting or washing away effector cells that become unbound from the functionalized wall, before or after capturing the image;
m) optionally, repeating steps j) to l), one or more times, with for each consecutive time an increasing centrifugal force;
n) optionally, collecting the effector cells that remain bound to the functionalized wall after the image was captured.

In an embodiment of the above-mentioned method the coating of the bottom surface with anti-fouling is optional. In an embodiment of the above-mentioned method the bottom surface is made of a material with anti-fouling properties. In an embodiment of the above-mentioned method the bottom surface does not comprise a coating with anti-fouling and/or is not made of a material with anti-fouling properties. In another embodiment of the above-mentioned method in step (b) the cells provided at a defined concentration are tumour cells such as for example tumour B cells.

In yet another embodiment, a method is provided of assessing cellular avidity of a cell with a functionalized wall comprising the steps of:
a) providing an avidity sample plate with one or more wells with a ceiling surface comprising a functionalized wall, wherein at least the bottom surface is coated with anti-fouling, wherein the avidity sample plate is optionally provided with an identifier, and, optionally provided with one or more sensors;
b) providing cells at a defined concentration;
c) optionally, and preferably, staining the cells;
d) introducing the cells, homogenized, to one or more wells of the avidity sample plate in the upright position and optionally allowing the cells to settle to the bottom of the avidity sample plate;
e) placing the avidity sample plate in the upside down position to allow the cells to settle on the functionalized wall of the avidity sample plate and incubating the cells with the functionalized wall, for a defined time;
f) optionally, capturing, with the avidity sample plate in the upside down position, an image of the cells;
g) placing the avidity sample plate in the upright position;
h) optionally, allowing the cells that did not bind to the functionalized wall to settle to the bottom of the avidity sample plate, and optionally, collecting or washing away said cells that settled to the bottom;
i) optionally, capturing an image of the cells bound to the functionalized wall, with the avidity sample plate in the upright position;
j) placing the avidity sample plate in the upright position in the centrifuge and centrifuging, at a defined centrifugal force, e.g. 1000×g, for a defined time;
k) capturing an image of the cells bound to the functionalized wall, with the avidity sample plate in the upright position;
l) optionally, collecting or washing away cells that become unbound from the functionalized wall, before or after capturing the image;
m) optionally, repeating steps j) to l), one or more times, with for each consecutive time an increasing centrifugal force;
n) optionally, collecting the cells that remain bound to the functionalized wall after the image was captured.

In yet another embodiment, a method is provided of assessing cellular avidity of a cell with a functionalized wall or a monolayer of target cells comprising the steps of:
a) providing an avidity sample plate with one or more wells with a ceiling surface comprising a functionalized wall or a monolayer of target cells, wherein at least the bottom surface is coated with anti-fouling, wherein the avidity sample plate is optionally provided with an identifier, and, optionally provided with one or more sensors;
b) providing cells at a defined concentration;
c) optionally, and preferably, staining the cells;
d) introducing the cells, homogenized, to one or more wells of the avidity sample plate in the upright position and optionally allowing the cells to settle to the bottom of the avidity sample plate;
e) placing the avidity sample plate in the upside down position to allow the cells to settle on the functionalized wall or the monolayer of target cells of the avidity sample plate and incubating the cells with the functionalized wall, for a defined time;
f) optionally, capturing, with the avidity sample plate in the upside down position, an image of the cells;
g) placing the avidity sample plate in the upright position;
h) optionally, allowing the cells that did not bind to the functionalized wall or the monolayer of target cells to settle to the bottom of the avidity sample plate, and optionally, collecting or washing away said cells that settled to the bottom;
i) optionally, capturing an image of the cells bound to the functionalized wall or the monolayer of target cells, with the avidity sample plate in the upright position;
j) placing the avidity sample plate in the upright position in the centrifuge and centrifuging, at a defined centrifugal force, e.g. 1000×g, for a defined time;
k) capturing an image of the cells bound to the functionalized wall or the monolayer of target cells, with the avidity sample plate in the upright position;
l) optionally, collecting or washing away cells that become unbound from the functionalized wall or the monolayer of target cells, before or after capturing the image;
m) optionally, repeating steps j) to l), one or more times, with for each consecutive time an increasing centrifugal force;
n) optionally, collecting the cells that remain bound to the functionalized wall or the monolayer of target cells after the image was captured.

In an embodiment of the above-mentioned method the coating of the bottom surface with anti-fouling is optional. In an embodiment of the above-mentioned method the bottom surface is made of a material with anti-fouling properties. In an embodiment of the above-mentioned method the bottom surface does not comprise a coating with anti-fouling and/or is not made of a material with anti-fouling properties. In another embodiment of the above-mentioned method in step (b) the cells provided at a defined concentration are tumour cells (such as for example tumour B cells) or effector cells.

Of course, as i.a. described elsewhere herein, these embodiments as described above utilizing a monolayer of target cells, or a functionalized wall may also include in the methods described additional molecules, e.g. small molecules or biologicals, as these may affect the cellular avidity between an (effector or tumour) cell and a monolayer of target cells or a functionalized wall as well. Such molecules may be any type of molecule suitable to affect, e.g. enhance, reduce or block, the interaction between the (effector or tumour) cell and the monolayer of target cells or the functionalized wall. For example, cell engagers may be of interest. Of course, the methods of the invention are not limited to the use of cell engagers, and other molecules, or combinations thereof, that may affect the interaction between an effector cell or a tumour cell and the monolayer of target cells or the functionalized wall may be used therein. Hence, in a further embodiment, additional molecules (e.g. cell engagers) and/or compounds are added before, during or after introducing the effector cells or tumour cells in the avidity sample plate. Such methods may be highly useful for screening molecules, e.g. by identifying molecules that are particular capable of enhancing, reducing or blocking binding of the cell to the monolayer of target cells or the functionalized wall.

In yet another embodiment, a method is provided of assessing cellular avidity of a cell with a functionalized wall or a monolayer of target cells comprising the steps of:
a) providing an avidity sample plate with one or more wells with a ceiling surface comprising a functionalized wall or a monolayer of target cells, wherein at least the bottom surface is coated with anti-fouling, wherein the avidity sample plate is optionally provided with an identifier, and, optionally provided with one or more sensors;
b) providing cells at a defined concentration;
c) optionally, and preferably, staining the cells;
d) introducing the cells, homogenized, to one or more wells of the avidity sample plate in the upright position and optionally allowing the cells to settle to the bottom of the avidity sample plate;
e) placing the avidity sample plate in the upside down position to allow the cells to settle on the functionalized wall or the monolayer of target cells of the avidity sample plate and incubating the cells with the functionalized wall, for a defined time;
f) optionally, capturing, with the avidity sample plate in the upside down position, an image of the cells;
g) placing the avidity sample plate in the upright position;
h) optionally, allowing the cells that did not bind to the functionalized wall or the monolayer of target cells to settle to the bottom of the avidity sample plate, and optionally, collecting or washing away said cells that settled to the bottom;
i) optionally, capturing an image of the cells bound to the functionalized wall or the monolayer of target cells, with the avidity sample plate in the upright position;
j) placing the avidity sample plate in the upright position in the centrifuge and centrifuging, at a defined centrifugal force, e.g. 1000×g, for a defined time;
k) capturing an image of the cells bound to the functionalized wall or the monolayer of target cells, with the avidity sample plate in the upright position;
l) optionally, collecting or washing away cells that become unbound from the functionalized wall or the monolayer of target cells, before or after capturing the image;
m) optionally, repeating steps j) to l), one or more times, with for each consecutive time an increasing centrifugal force;
n) optionally, collecting the cells that remain bound to the functionalized wall or the monolayer of target cells after the image was captured.

In an embodiment of the above-mentioned method the coating of the bottom surface with anti-fouling is optional. In an embodiment of the above-mentioned method the bottom surface is made of a material with anti-fouling properties. In an embodiment of the above-mentioned method the bottom surface does not comprise a coating with anti-fouling and/or is not made of a material with anti-fouling properties. In another embodiment of the above-mentioned method in step (b) the cells provided at a defined concentration are tumour cells (such as for example tumour B cells) or effector cells.

In an embodiment a new avidity sample plate is used. In another embodiment the avidity sample plate is prepared by rehydration in the following way. A clean and dried avidity sample plate is taken out of a dry incubator and one or more wells are rehydrated with buffer, such as for example PBS.

Next, the surfaces of the wells of the avidity sample plate are functionalized or provided with a monolayer of cells. In a preferred embodiment at least the ceiling surface of the wells is functionalized or provided with a monolayer of cells. In an embodiment the functionalisation is done by coating the one or more surfaces with one or more polypeptides, such as for example a glycoprotein such as fibronectin. Instead of fibronectin, a monolayer of cells such as a monolayer of stromal cells could be used. In a specific embodiment 1-50 μg/ml, preferably 5-25 μg/ml, more preferably about 10 μg/ml of a polypeptide, for example fibronectin, is applied. Application is dependent on the polypeptide used and can be done as known in the art, for example for fibronectin for a minimum of 1 hour at 37° C. in a dry incubator.

Before use, the sample plate is taken and one or more wells are washed once with fresh buffer, such as for example PBS. This is followed by blocking with a suitable blocking agent, e.g. BSA, to avoid unspecific binding. The conditions for incubation are dependent on the agent used for blocking and are known in the art, for example 4% (w/w) BSA for 10 minutes in a dry incubator.

After a defined time, e.g. 10 minutes, one or more wells of the avidity sample plate are washed at least once with buffer followed by another wash with avidity assay medium, e.g. culture medium such as for example complete (with serum) RPMI medium, for a defined time, e.g. 10 seconds for each wash.

Before performing the avidity assay, i.e. assessing the cellular avidity of cells and the functionalized wall or the monolayer of cells, cells, e.g. B cells, were harvested and stained with for example a dye, such as for example CellTrace™ Far Red Dye (Thermo Fisher) essentially according to the instructions of the manufacturer at an initial defined density, e.g. 20×10⁶ cells/ml.

In an embodiment during the preparation of the avidity sample plate (e.g. incubation with fibronectin or a monolayer of stromal cells), the cells are prepared (e.g. the cells are harvested and stained as described above).

In an embodiment half of the stained population is kept in avidity assay medium without additional molecule, while the other half of the stained population is kept in avidity assay medium with an additional molecule, e.g. a small molecule kinase inhibitor.

Conditions (e.g. concentrations, time, temperature, etc) depend on the additional molecule to be applied and are known in the art. For example, a small molecule kinase inhibitor can be used at a final concentration of 100 nM for 1 hour at 37° C.

In an embodiment this is followed by at least one wash and final resuspension in avidity assay medium containing the additional molecule, e.g. the small molecule kinase inhibitor.

In an embodiment the stained cells with and without additional molecule are introduced into separate wells of the avidity sample plate with and without anti-IgM stimulation. Preferably, the anti-IgM is introduced into the wells simultaneously with the cells. Preferably, the final concentration of cells, e.g. B cells in each well is at a defined concentration, e.g. 10 million/ml for the B cells and the final concentration is e.g. 100 ng/ml for the anti-IgM molecule. Next, after a defined incubation time, e.g. 10 minutes, avidity is measured as described herein, e.g. by placing the avidity sample plate in the centrifuge and centrifuging, at a defined centrifugal force, e.g. 1000×g, for a defined time. Preferably, the avidity sample plate is placed in the upright position in the centrifuge and centrifuged, at a defined centrifugal force, e.g. 1000×g, for a defined time.

The present invention also relates to a method of assessing cellular avidity of an effector cell and a target cell comprising the steps of:
a) providing an avidity sample plate with one or more wells with a ceiling surface coated with target cells;
b) placing the avidity sample plate in an upright position;
c) introducing effector cells to one or more wells of the avidity sample plate in the upright position;
d) placing the avidity sample plate in the upside down position to allow the effector cells to settle on the target cells attached to the ceiling of the one or more wells of the avidity sample plate and incubating the effector cells with the target cells for a defined time;
e) capturing an image of the effector cells and the target cells;
f) placing the avidity sample plate in the upright position in a centrifuge and centrifuging the avidity sample plate;
g) capturing an image of the effector cells bound to the target cells;
h) optionally, repeating steps (f) and (g), one or more times, with for each consecutive time an increasing centrifugal force(s);
i) determining how many effector cells are bound to the target cells after centrifugation.

All embodiments and features as described in the present application may also apply to the just described method of assessing cellular avidity of an effector cell and a target cell.

In an embodiment at least the bottom surface of the one or more wells of the avidity sample plate is coated with anti-fouling and/or made of material that has anti-fouling properties. In an embodiment the bottom surface does not have any anti-fouling properties. In an embodiment the avidity sample plate is provided with an identifier and/or provided with one or more sensors. In an embodiment the target cells are labelled. In an embodiment the ceiling surface comprises a coating agent suitable for attachment of the target cells. In an embodiment the ceiling surface is coated with a monolayer of target cells. In an embodiment the target cells are cultured, with the avidity sample plate in either upside down position or upright position, in a controlled incubator. In an embodiment the ceiling surface with attached target cells is blocked with a blocking agent. In an embodiment the target cells are washed, with the avidity sample plate in the upright position, to remove target cells that did are loosely attached to the ceiling surface. In an embodiment an image of the target cells attached to the ceiling surface is acquired.

As described above, the avidity sample plate is placed in an upright position.

In an embodiment the effector cells that are introduced to the one or more wells of the avidity sample plate are provided at a defined concentration. In an embodiment the effector cells are labelled, for example fluorescently labelled. In an embodiment the effector cells are homogenized before introduction. In an embodiment the effector cells are introduced together with one or more additional molecules, such as for example one or more cell engagers, to the one or more wells of the avidity sample plate. The effector cells and the one or more additional molecules may be combined before introduction and introduced as a mixture. Alternatively, the effector cells and the one or more additional molecules may be introduced consecutively in any order. In an embodiment the effector cells and/or one or more additional molecules are first allowed to settle to the bottom of the avidity sample plate before placing the avidity sample plate in the upside down position to allow the effector cells and/or one or more additional molecules to settle on the target cells attached to the ceiling of the one or more wells of the avidity sample plate.

After placing the avidity sample plate in the upside down position to allow the effector cells to settle on the target cells attached to the ceiling of the one or more wells of the avidity sample plate and incubating the effector cells with the target cells for a defined time, an image of the effector cells and the target cells is captured. In an embodiment the image is captured with the avidity sample plate in the upside down position.

In an embodiment, before placing the avidity sample plate in the upright position in a centrifuge and centrifuging the avidity sample plate, any inlets and/or outlets in the one or more wells of the avidity sample plate are sealed or capped. In an embodiment, before placing the avidity sample plate in the upright position in a centrifuge and centrifuging the avidity sample plate, the effector cells that did not bind to the target cells are allowed to settle to the bottom of the avidity sample plate, and optionally, said effector cells that settled to the bottom are collected or washed away.

In an embodiment the centrifuging is done at a defined centrifugal force, e.g. 1000×g, for a defined time.

After centrifuging, an image of the effector cells bound to the target cells is captured. In an embodiment an image of the effector cells bound to the monolayer of target cells is captured. In an embodiment an image is captured with the avidity sample plate in the upright position.

The centrifuging step and the image capturing step may be repeated one or more times, with for each consecutive time an increasing centrifugal force(s). In an embodiment the centrifugal force is kept constant, but the centrifugation time is increased with each consecutive centrifuging step. In an embodiment the effector cells that remain bound to the target cells after the centrifuging step and the image capturing step are collected. In an embodiment it is determined how many effector cells are bound to the target cells after the centrifuging step and the image capturing step. In an embodiment the determination of the number of effector cells bound to the target cells after the centrifuging step and the image capturing step is done by counting the effector cells bound to target cells pre and post centrifugation. In an embodiment the image capturing step is done by using a fluorescent microscope. In an embodiment the images captured are brightfield and fluorescence images. In an embodiment post centrifugation images are provided after each centrifugation step. In an embodiment the percentage of effector cells bound after each centrifugation step is quantified by comparing the number of effector cells present in the pre vs post centrifugation images. To get the data, the images may be analysed using custom in-house cell detection image analysis software. The percentage of effector cells bound in the pre vs post centrifugation conditions may be a measure of cellular avidity. Cellular avidity induced by one or more additional molecules, for example cell engagers, can be determined by subtracting the fraction of bound effector cells in the absence of the one or more additional molecules from the fraction of bound effector cells in the presence of the one or more additional molecules.

The present invention also relates to a method of assessing cellular avidity of a cell and a polypeptide comprising the steps of:
a) providing an avidity sample plate with one or more wells with a ceiling surface coated with a polypeptide;
b) placing the avidity sample plate in an upright position;
c) introducing cells to one or more wells of the avidity sample plate in the upright position;
d) placing the avidity sample plate in the upside down position to allow the cells to settle on the polypeptide attached to the ceiling of the one or more wells of the avidity sample plate and incubating the cells with the polypeptide for a defined time;
e) capturing an image of the cells and the polypeptide;
f) placing the avidity sample plate in the upright position in a centrifuge and centrifuging the avidity sample plate;
g) capturing an image of the cells bound to the polypeptide;
h) optionally, repeating steps (f) and (g), one or more times, with for each consecutive time an increasing centrifugal force(s);
i) determining how many cells are bound to the polypeptide after centrifugation.

All embodiments and features as described in the present application may also apply to the just described method of assessing cellular avidity of a cell and a polypeptide. The polypeptide may be a protein, binding molecule (e.g. antibody or part or derivative thereof), peptide, antigen, ligand, receptor, compound, to name just a few. In an embodiment two or more, three or more, four or more, five or more different polypeptides may be used.

### EMBODIMENTS

1. A method of assessing cellular avidity of an effector cell and a target cell comprising the steps of:
   a) providing target cells at a defined concentration;
   b) optionally, staining the target cells;
   c) providing an avidity sample plate with one or more wells with a ceiling surface coated for attachment of target cells, wherein optionally at least the bottom surface is coated with anti-fouling or optionally at least has antifouling properties, wherein the avidity sample plate is optionally provided with an identifier, and, optionally provided with one or more sensors;
   d) introducing the target cells, homogenized, to one or more wells of the avidity sample plate for obtaining a monolayer of target cells, with the avidity sample plate in the upright position;
   e) placing the avidity sample plate in an upside down position, thereby allowing the target cells to settle to the ceiling surface and attach thereto to form a monolayer;
   f) optionally, culturing the target cells, with the avidity sample plate in either upside down position or upright position, in a controlled incubator;
   g) optionally, blocking the ceiling surface with attached target cells with a blocking agent;
   h) placing the avidity sample plate in the upright position, optionally in a centrifuge and centrifuge to spin down target cells that did not attach to the ceiling surface;
   i) optionally, washing the cells with the avidity sample plate in the upright position, to remove target cells that did not attach to the ceiling surface;
   j) optionally, acquire an image of the monolayer of target cells attached to the ceiling surface.
   k) optionally, checking the quality of monolayer of target cells based on the image of the monolayer;
   l) providing effector cells at a defined concentration;
   m) optionally, and preferably, staining the effector cells;
   n) introducing the effector cells, homogenized, to one or more wells of the avidity sample plate in the upright position and optionally allowing the effector cells to settle to the bottom of the avidity sample plate;
   o) placing the avidity sample plate in the upside down position to allow the effector cells to settle on the monolayer of target cells attached to the ceiling of the avidity sample plate and incubating the effector cells with the target cells, for a defined time;
   p) optionally, capturing, with the avidity sample plate in the upside down position, an image of the effector cells and the monolayer of target cells;
   q) placing the avidity sample plate in the upright position;
   r) optionally, allowing the effector cells that did not bind to the target cells to settle to the bottom of the avidity sample plate, and optionally, collecting or washing away said effector cells that settled to the bottom;
   s) optionally, capturing an image of the effector cells bound to the monolayer of target cells, with the avidity sample plate in the upright position;
   t) placing the avidity sample plate in the upright position in the centrifuge and centrifuging, at a defined centrifugal force, e.g. 1000×g, for a defined time;
   u) capturing an image of the effector cells bound to the monolayer of target cells, with the avidity sample plate in the upright position;
   v) optionally, collecting or washing away effector cells that become unbound from the target cells, before or after capturing the image;
   w) optionally, repeating steps t) to v), one or more times, with for each consecutive time an increasing centrifugal force(s);
   x) optionally, collecting the effector cells that remain bound to the target cells after the image was captured.
2. The method in accordance with embodiment 1, wherein images are captured using a microscope from below the avidity sample plate.
3. The method in accordance with embodiment 1, wherein images are captured using a microscope from above of the avidity sample plate.
4. The method in accordance with embodiment 2 or embodiment 3, wherein the microscope is provided with multiple objectives, one of which can be selected for capturing an image.
5. The method in accordance with any one of embodiments 1-4, wherein the avidity sample plate is made from a polymer, such as polystyrene.
6. The method in accordance with any one of embodiments 1-5, wherein the avidity sample plate is in a 48-well, 96-well, or 384-well format.
7. The method in accordance with any one of embodiments 1-6, wherein the ratio of target cells to effector cells is in the range from 1:1 to 100:1, preferably about 10:1.
8. The method in accordance with any one of embodiments 1-7, wherein the collection step of the effector cells that remain bound to the target cells comprises subjecting the effector cells bound to the attached target cells to a trypsin treatment and/or resuspension step.
9. The method in accordance with any one of embodiments 1-8, wherein multiple effector cell types or clones are provided, wherein each of the effector cell types or clones is provided to a different well.
10. The method in accordance with embodiment 1-8, wherein multiple effector cell types or clones are provided, wherein each of the effector cell types or clones is provided with a different staining, and wherein multiple effector cell types or clones are combined and provided to individual wells.
11. The method in accordance with any one of embodiments 1-10, wherein the target cells are stained as well, which staining is different from the staining of the effector cells.
12. The method in accordance with any one of embodiments 1-11, wherein different target cells are provided, optionally including control target cells, wherein the different target cells are provided to separate wells.
13. The method in accordance with any one of embodiments 1-12, wherein control effector cells are provided in addition.
14. The method in accordance with embodiment 13, wherein the control effector cells are provided to separate wells.
15. The method in accordance with embodiment 13, wherein the control effector cells are included in each well, wherein the control effector cells are differently stained.
16. The method in accordance with embodiment 15, wherein the ratio of control effector cells to effector cells in a well is in the range of from 1:1 to 1:10.
17. Method in accordance with embodiment 1-16, wherein the images are analysed.
18. The method in accordance with any one of embodiments 17, wherein analysing the captured images comprising determining the number of effector cells in captured images.
19. The method in accordance with embodiment 17 or 18, wherein the determined numbers are used to calculate a cellular avidity score for each well.
20. The method in accordance with embodiment 19, wherein the cellular avidity score is the ratio of the number of effector cells that remained bound to the number of effector cells initially provided.
21. The method in accordance with any one of embodiments 1-20, wherein immune synapses are detected.
22. The method in accordance with embodiment 21, wherein analysing the captured images comprises determining the number of effector cells that formed a synapse.
23. The method in accordance with embodiment 22, wherein a cellular avidity score is determined based on the determined number of effector cells that formed a synapse.
24. The method in accordance with any one of embodiments 1-23, wherein the duration of each step is recorded.
25. The method in accordance with any one of embodiments 1-24, wherein the duration of each step is controlled.
26. The method in accordance with any one of embodiments 1-25, wherein the avidity sample plate is provided with one or more sensors and the one or more sensors are selected from an accelerometer, a thermometer, a humidity sensor, a CO₂ sensor, and wherein in the method sensor readings are registered in time.
27. The method in accordance with any one of embodiments 1-26, wherein additional molecules (e.g. cell engagers) and/or compounds are added before, during or after introduction of the effector cells.
28. An cellular avidity measuring system comprising a centrifuge unit, an avidity measuring unit, an avidity sample plate holder, and one or more avidity sample plates that fit in the avidity sample plate holder.
29. The cellular avidity measuring system in accordance with embodiment 28, wherein the avidity measuring system further comprises an incubator unit.
30. The cellular avidity measuring system according to embodiment 28 or 29, wherein the avidity sample plate holder is compatible with the centrifuge unit, the avidity measuring unit, and, optionally, the incubator unit.
31. The cellular avidity measuring system according to embodiment 28 to 30, wherein the avidity measuring unit comprises a microscope and a liquid handling unit.
32. The cellular avidity measuring system according to embodiment 31, wherein the microscope is a fluorescence microscope with one more fluorescent channels.

### EXAMPLES

### Example 1

A monolayer of target Nalm6 (CD19+) cells (obtained from ATCC, product nr. CRL-1567) was brought in contact with IL-2 stimulated primary human effector T cells purified from buffy coat, which were transduced with CAR-FMC63 anti-CD19 (Kramer, A M; (2017) Delineating the impact of binding-domain affinity and kinetic properties on Chimeric Antigen Receptor T cell function. Doctoral thesis, UCL (University College London)). After removal of free and unbound cells by washing and centrifugation, cells remaining bound were assessed with microscopy.

### Monolayer Seeding

Nalm6 (CD19+) target cells were used, which were seeded as a monolayer to the ceiling of a 400 μm tall channel slide (μ-Slides obtained from Ibidi, Cat. No: 80176). First, cells were counted and resuspended in serum-free medium at a concentration of approximately 30×10⁶ cells/mL in order to acquire a confluence close to 100%. Next, 100 μL of the resuspended cells was pipetted into the inlet of the channel slide and introduced in the channel by tilting the slide for few seconds until it reached the outlet of the channel slide. The channel slide was then placed horizontally so that the 100 μL filled the entire volume of the channel. The channel slide was flipped immediately, and incubated upside down in a humidity, temperature, and CO₂ controlled incubator for 30 minutes. After incubation, the medium was exchanged by first applying 60 μL of serum-containing medium into the inlet and subsequently withdrawing 60 μL from the outlet. This process was repeated four times. The slide was placed back upside down in the controlled incubator and incubated for another 30 minutes. Next, the medium was exchanged with PBS containing 0,5-1 μM CellTrace Violet (ThermoFisher #C34557) and incubated for 15 minutes in the incubator. Finally, the staining solution was exchanged by first applying 60 μL of the serum-containing medium into the inlet and subsequently withdrawing 60 μL from the outlet. This process was repeated four times.

### Effector Cell Preparation

In preparation of subsequent steps, primary T cells transduced with the anti-CD19 CAR-FMC63 were cultured. Cells were counted and viability was tested according to standard protocol. Next, 3×10⁶ cells/mL were stained with PBS containing 1 μM CellTrace™ Green CMFDA (Thermo Fisher #C2925) and incubated for 15 minutes in the incubator. Finally, the primary T cells were pelleted and resuspended in complete medium.

### Effector Cell Binding

The channel inlet and outlet were filled with plugs prior to centrifugation. The channel slide containing the seeded Nalm6 target cells was placed upright into a custom adaptor for the centrifuge bucket and spun for two minutes at 1000×g. Centrifugation removes fractions of the cells that were not well attached to the glass. Next, the slide was removed from the centrifuge. First, 100 μL of the effector cell suspension was pipetted into the inlet after which 100 μL was withdrawn from the outlet in order to obtain a homogenous distribution of effector cells throughout the length of the channel. The slide containing the target and effector cells was held upside down (to allow the effector cells and Nalm6 cells on the ceiling to interact) and incubated for five minutes. After incubation, three locations of the channel were imaged (one in the centre and one close to the inlet/outlet) using a fluorescent microscope. Each location was imaged in the brightfield, Green, and Violet channel. The slide was placed back in the centrifuge using the custom adapter and spun for two minutes at 1000×g. Finally, the channel slide was removed from the centrifuge and imaged at the same locations and using the same channels. It was observed that about 50% of the cells remained bound to the target cell after centrifugation. In general, low background binding is observed with Nalm6 cells when applying the centrifuge procedure, which usually is in the range of 5-15%.

### Example 2

### Assessment of Cellular Avidity of Effector Cells and Target Cells in the Presence of a Cell Engager

Cellular avidity of effector cells and target cells was measured in the presence of a cell engager by using the methods for assessing cellular avidity as described herein.

Nalm6 (CD19+) cells (obtained from ATCC, product nr. CRL-1567) were used as target cells. The Nalm6 (CD19+) cells were grown, harvested and resuspended in serum free RPMI1664 medium. Hereafter, 6-channel slides (Ibidi, product number 80606-90) were seeded with 0.9×10⁶ cells/ml in each channel and the slides were flipped and incubated upside down for 30 minutes in humid incubator at 37° C.

After incubation, the slides were flipped and the medium in each channel was exchanged twice with 150 μl RPMI1664 medium supplemented with 10% fetal calf serum (FCS). The slides were incubated upside down again for 30 minutes in humid incubator at 37° C.

Thereafter, to test monolayer integrity, the slides were flipped to put them in an upright position and they were centrifuged at 10 relative centrifugal force (rcf) for 2 minutes, washed and a visual check of monolayer integrity was performed using a table top microscope. The slides were stored upside down in humid incubator at 37° C. until use.

Healthy donor T cells were used as effector cells. The T cells were stained in phosphate buffered saline (PBS) with 5 μM CellTrace™ Far Red for 15 minutes at 37° C. and washed with 10 ml of PBS. The T cells were subsequently resuspended in RPMI1664 medium supplemented with 10% FCS.

Next, the antibody and bi-specific T cell engager called Blinatumomab (Invivogen) was added to the T cells at the following concentrations: 1 ng/ml, 10 ng/ml, 100 ng/ml or 1000 ng/ml (with ng/ml is meant ng Ab/ml cell suspension). As a control, no antibody was added. The obtained mixtures were added directly to the channels. After all channels were filled, the slides containing the target and effector cells were flipped upside down to allow the T cells and cell engager and Nalm6 cells on the ceiling to interact and incubation for 10 minutes at 37° C. started.

After incubation, the number of T cells on the monolayer were imaged with the slides being upside down and then the slides were flipped again to put them in an upright position.

Thereafter, the slides were put into a centrifuge in an upright position and a force in a direction away from the target cell monolayer was exerted by centrifuging the slides at 10 rcf for 2 minutes.

After centrifugation, the slides were taken out of the centrifuge and images were taken of the T cells that remained attached to the target cell monolayer. Monolayer bound T cells were counted using in-house made software. The percentage of T cells that remained attached was determined, while the T cells that were attached before exerting the force were used to set the baseline.

The results are shown in Table 1. They show that the method according to the invention and as described herein can successfully be used to measure cellular avidity of effector cells and target cells in the presence of a cell engager. The results further show that increasing the concentration of the cell engager increases the number of effector cells that remain attached to the target cell monolayer after exertion of a centrifugal force which is directed away from the target cell monolayer.

### Example 3

### Assessment of Cellular Avidity of Effector Cells and Target Cells in the Presence of a Cell Engager

Cellular avidity of effector cells and target cells was measured in the presence of a cell engager by using the methods for assessing cellular avidity as described herein. The experiment was essentially similar as the experiment described in Example 2.

Nalm6 (CD19+) cells (obtained from ATCC, product nr. CRL-1567) were used as target cells. The Nalm6 (CD19+) cells were grown, harvested and resuspended in serum free RPMI1664 medium. Hereafter, 6-channel flow cells (having a channel volume of 30 μl and a surface area of 0.6 cm²) were seeded with 30×10⁶ cells/ml in each channel and the flow cells were flipped and incubated upside down for 60 minutes in a humid incubator at 37° C. To promote monolayer cell adhesion, the channels were precoated with 0.02 mg/ml of poly-L-lysine.

The flow cells were stored upside down in a humid incubator at 37° C. until use and immediately before introducing the effector cells and the cell engager, target cell monolayers were force tested and each channel was washed to remove any detached target cells.

Jurkat E6.1 cells were used as effector cells. The cells were stained for imaging using 5 UM of the fluorescent dye CellTrace™ Far Red in phosphate buffered saline (PBS) for 15 minutes at 37° C. and washed with 10 ml of PBS. After staining, the cells were resuspended in RPMI1664 medium supplemented with 10% FCS at a concentration of 0.5×10⁶/ml and placed at 37° C.

The bispecific antibody Blinatumomab (Blincyto®) was used as a cell engager, while a control Bi-specific T-cell engager containing an identical CD3-targeting scFv as Blinatumomab but with a target arm directed against bacteria-derived β-galactosidase protein (absent in Nalm6 (CD19+) cells) was used as a negative control cell engager. As another control, no antibody was added.

Immediately before use, the effector cells were mixed with the appropriate cell engager dilution (2×10⁻⁵ nM, 2×10⁻⁴ nM, 2×10⁻³ nM, 2×10⁻² nM, 2×10⁻¹ nM, 2×10⁰ nM, 2×10¹ nM, 2×10² nM or 2×10³ nM) and introduced into the target cell-containing channels of the 6-channel flow cells and incubated for 2, 5, 10, 20 or 40 minutes after flipping the flow cells upside down to allow the effector cells and cell engager and target cells on the ceiling to interact.

After incubation, the number of effector cells on the target cell monolayer were imaged using a fluorescence microscope with the flow cells being upside down. In this step pre-centrifugation brightfield and fluorescence images were acquired for each channel. After pre-centrifugation image collection was applied, the flow cells were flipped again to put them in an upright position.

Thereafter, the flow cells were placed into a centrifuge in an upright position and a force in a direction away from the target cell monolayer was exerted by centrifuging the flow cells at 1000×g for 2 minutes.

After centrifugation, the flow cells were taken out of the centrifuge and brightfield and fluorescence images were acquired of the effector cells that remained attached to the target cell monolayer.

The percentage of effector cells bound after the centrifugation step (i.e. the force application step) was quantified by comparing the number of effector cells present in the pre vs post-force application fluorescence images for all channels. Fluorescence images were analysed using custom in-house cell detection image analysis software.

The avidity value was calculated as the channel averaged fraction of effector cells which remain bound to the monolayer after force application. The cell engager-induced cell avidity signal was determined by subtracting the fraction of bound effector cells of the no cell engager control channel from the fraction of bound effector cells of each cell engager-containing channel.

The results of the experiments (data not shown) demonstrate a Blinatumomab dose-dependent response in cell avidity, confirming the applicability of the methods as described herein for investigating cell engager-mediated cell avidity. The highest cell avidity signal was observed at a Blinatumomab concentration of 20 nM for all the incubation times tested. At higher concentrations, the avidity signal remained stable or decreased.

The results further show that increasing incubation time resulted in an increase in the avidity signal, demonstrating time-dependent behaviour of cell engager-specific cell engagement.

Based on the above, it can be concluded that the methods as described herein can determine cell engager-specific avidity with high precision, sensitivity and reproducibility over a wide range of cell engager concentrations.

Moreover, it can be concluded that the methods as described herein allow for highly controlled force application and simultaneous, high throughput analysis of cell avidity between effector cells and target cells in the presence of cell engagers and consequently are highly suitable for cell engager candidate screening in high throughput format, with high sensitivity and precision.

TABLE 1
Cellular avidity measurements of effector cells and
target cells in the presence of a cell engager

| | Cell engager concentration (in ng/ml) | | | | |
|---|---|---|---|---|---|
| | 0 | 1 | 10 | 100 | 1000 |
| Slide 1 (% of T cells remaining | 29.0 | 29.7 | 40.0 | 55.2 | 68.8 |
| attached) | | | | | |
| Slide 1 (% of T cells remaining | 28.4 | 31.0 | 40.3 | 52.1 | 72.2 |
| attached) | | | | | |
| Slide 2 (% of T cells remaining | 24.1 | 26.8 | 35.7 | 52.3 | 64.1 |
| attached) | | | | | |
| Slide 2 (% of T cells remaining | 23.6 | 31.2 | 37.2 | 48.9 | 65.7 |
| attached) | | | | | |

1. A method of assessing cellular avidity of an effector cell and a target cell comprising the steps of:
a) providing target cells at a defined concentration;
b) optionally, staining the target cells;
c) providing an avidity sample plate with one or more wells with a ceiling surface coated for attachment of target cells, wherein optionally at least the bottom surface is coated with anti-fouling, wherein the avidity sample plate is optionally provided with an identifier, and, optionally provided with one or more sensors;
d) introducing the target cells, homogenized, to one or more wells of the avidity sample plate for obtaining a monolayer of target cells, with the avidity sample plate in the upright position;
e) placing the avidity sample plate in an upside down position, thereby allowing the target cells to settle to the ceiling surface and attach thereto to form a monolayer;
f) optionally, culturing the target cells, with the avidity sample plate in either upside down position or upright position, in a controlled incubator;
g) optionally, blocking the ceiling surface with attached target cells with a blocking agent;
h) placing the avidity sample plate in the upright position, optionally in a centrifuge and centrifuge to spin down target cells that did not attach to the ceiling surface;
i) optionally, washing the cells with the avidity sample plate in the upright position, to remove target cells that did not attach to the ceiling surface;
j) optionally, acquire an image of the monolayer of target cells attached to the ceiling surface.
k) optionally, checking the quality of monolayer of target cells based on the image of the monolayer;
l) providing effector cells at a defined concentration;
m) optionally, and preferably, staining the effector cells;
n) introducing the effector cells, homogenized, to one or more wells of the avidity sample plate in the upright position and optionally allowing the effector cells to settle to the bottom of the avidity sample plate;
o) placing the avidity sample plate in the upside down position to allow the effector cells to settle on the monolayer of target cells attached to the ceiling of the avidity sample plate and incubating the effector cells with the target cells, for a defined time;
p) optionally, capturing, with the avidity sample plate in the upside down position, an image of the effector cells and the monolayer of target cells;
q) placing the avidity sample plate in the upright position;
r) optionally, allowing the effector cells that did not bind to the target cells to settle to the bottom of the avidity sample plate, and optionally, collecting or washing away said effector cells that settled to the bottom;
s) optionally, capturing an image of the effector cells bound to the monolayer of target cells, with the avidity sample plate in the upright position;
t) placing the avidity sample plate in the upright position in the centrifuge and centrifuging, at a defined centrifugal force, e.g. 1000×g, for a defined time;
u) capturing an image of the effector cells bound to the monolayer of target cells, with the avidity sample plate in the upright position;
v) optionally, collecting or washing away effector cells that become unbound from the target cells, before or after capturing the image;
w) optionally, repeating steps t) to v), one or more times, with for each consecutive time an increasing centrifugal force(s);
x) optionally, collecting the effector cells that remain bound to the target cells after the image was captured.

2. The method in accordance with claim 1, wherein images are captured using a microscope from below the avidity sample plate or wherein images are captured using a microscope from above of the avidity sample plate.

3. The method in accordance with claim 2, wherein the microscope is provided with multiple objectives, one of which can be selected for capturing an image.

4. The method in accordance with claim 1, wherein the avidity sample plate is in a 48-well, 96-well, or 384-well format.

5. The method in accordance with claim 1, wherein the collection step of the effector cells that remain bound to the target cells comprises subjecting the effector cells bound to the attached target cells to a trypsin treatment and/or resuspension step.

6. The method in accordance with claim 1, wherein multiple effector cell types or clones are provided, wherein each of the effector cell types or clones is provided to a different well.

7. The method in accordance with claim 1, wherein multiple effector cell types or clones are provided, wherein each of the effector cell types or clones is provided with a different staining, and wherein multiple effector cell types or clones are combined and provided to individual wells.

8. The method in accordance with claim 1, wherein different target cells are provided, optionally including control target cells, wherein the different target cells are provided to separate wells.

9. The method in accordance with claim 1, wherein the images are analysed.

10. The method in accordance with claim 9, wherein analysing the captured images comprising determining the number of effector cells in captured images.

11. The method in accordance with claim 10, wherein the determined numbers are used to calculate a cellular avidity score.

12. The method in accordance with claim 1, wherein immune synapses are detected.

13. The method in accordance with claim 1, wherein the avidity sample plate is provided with one or more sensors and the one or more sensors are selected from an accelerometer, a thermometer, a humidity sensor, a CO₂ sensor, and wherein in the method sensor readings are registered in time.

14. An cellular avidity measuring system comprising a centrifuge unit, an avidity measuring unit, an avidity sample plate holder, and one or more avidity sample plates that fit in the avidity sample plate holder, and wherein optionally, the avidity measuring system further comprises an incubator unit.

15. The cellular avidity measuring system according to claim 14, wherein the avidity sample plate holder is compatible with the centrifuge unit, the avidity measuring unit, and, optionally, the incubator unit; and/or wherein the avidity measuring unit comprises a microscope and a liquid handling unit.
